SEQUENCE LISTING (28 sequences)

SEQ ID NO: 1 - protein, 443 aa, Synthetic construct
  source 1..443: note = Dostarlimab Heavy Chain
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Synthetic construct
  source 1..214: note = Dostarlimab Light Chain
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Synthetic construct
  source 1..447: note = Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Synthetic construct
  source 1..218: note = Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 440 aa, Synthetic construct
  source 1..440: note = Nivolumab Heavy Chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, Synthetic construct
  source 1..214: note = Nivolumab Light Chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, Synthetic construct
  source 1..444: note = Cemiplimab Heavy Chain
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, Synthetic construct
  source 1..214: note = Cemiplimab Light Chain
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Synthetic construct
  source 1..448: note = Atezolizumab Heavy Chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Synthetic construct
  source 1..214: note = Atezolizumab Light Chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Synthetic construct
  source 1..450: note = Avelumab Heavy Chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Synthetic construct
  source 1..216: note = Avelumab Light Chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 451 aa, Synthetic construct
  source 1..451: note = Durvalumab Heavy Chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 215 aa, Synthetic construct
  source 1..215: note = Durvalumab Light Chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 448 aa, Synthetic construct
  source 1..448: note = Ipilimumab Heavy Chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Synthetic construct
  source 1..215: note = Ipilimumab Light Chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, Synthetic construct
  source 1..446: note = elatlimab Heavy Chain
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 214 aa, Synthetic construct
  source 1..214: note = Relatlimab Light Chain
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 441 aa, Synthetic construct
  source 1..441: note = Encelimab Heavy Chain
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 219 aa, Synthetic construct
  source 1..219: note = Encelimab Light Chain
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, Synthetic construct
  source 1..5: note = Dostarlimab CDRH1
SYDMS

SEQ ID NO: 22 - protein, 17 aa, Synthetic construct
  source 1..17: note = Dostarlimab CDRH2
TISGGGSYTYYQDSVKG

SEQ ID NO: 23 - protein, 7 aa, Synthetic construct
  source 1..7: note = Dostarlimab CDRH3
PYYAMDY

SEQ ID NO: 24 - protein, 11 aa, Synthetic construct
KASQDVGTAVA

SEQ ID NO: 25 - protein, 7 aa, Synthetic construct
  source 1..7: note = Dostarlimab CDRL2
WASTLHT

SEQ ID NO: 26 - protein, 9 aa, Synthetic construct
  source 1..9: note = Dostarlimab CDRL3
QHYSSYPWT

SEQ ID NO: 27 - protein, 116 aa, Synthetic construct
  source 1..116: note = Dostarlimab VH
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 28 - protein, 107 aa, Synthetic construct
  source 1..107: note = Dostarlimab VL
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

(19) United States
(12) Patent Application Publication
(10) Pub. No.: US 2026/0083839 A1
(43) Pub. Date: Mar. 26, 2026

(54) METHODS OF TREATING NON-SMALL CELL LUNG CANCER WITH ANTI-PD-1-ANTIBODIES

(71) Applicant: Tesaro, Inc., Wilmington, DE (US)

(72) Inventors: Martin HUBER, Boston, MA (US); Niranjan YANAMANDRA, Landsdale, PA (US); Allene DIAZ, Boston, MA (US); Mary Lynne HEDLEY, Lexington, MA (US); Julie Ann KRUEGER, North Wales, PA (US); Timothy R. CROSSMAN, Stevenage (GB); Hesham A. ABDULLAH, Collegeville, PA (US)

(21) Appl. No.: 19/113,681

(22) PCT Filed: Sep. 22, 2023
(86) PCT No.: PCT/US2023/074816

§ 371 Date: Mar. 20, 2025

Related U.S. Application Data
(60) Provisional application No. 63/410,022, filed on Sep. 26, 2022.
(60) Provisional application No. 63/426,527, filed on Nov. 18, 2022.
(60) Provisional application No. 63/453,338, filed on Mar. 20, 2023.
(60) Provisional application No. 63/579,094, filed on Aug. 28, 2023.

(51) Int. Cl. A61K 39/395; A61K 31/282; A61K 31/519; A61K 33/243; A61K 39/00; A61P 35/00; C07K 16/28

(52) U.S. Cl. CPC A61K 39/3955; A61K 31/282; A61K 31/519; A61K 33/243; A61P 35/00; C07K 16/2818; A61K 2039/505; A61K 2039/54; A61K 2039/545

(57) ABSTRACT

The disclosure provides a method of treating non-small cell lung cancer (NSCLC) in a patient previously treated with an immune checkpoint inhibitor by the administration of dostarlimab or a biosimilar thereof.

### FIELD

The present disclosure generally relates to methods of treating non-small cell lung cancer (NSCLC) in a patient previously treated with an immune checkpoint inhibitor by the administration of dostarlimab or a biosimilar thereof.

### BACKGROUND

Lung cancer is the most common cause of cancer mortality globally and the second most common cancer in both men and women. About 13% of all new cancers in the United States (US) are lung cancer. The two major forms of lung cancer are non-small cell lung cancer (NSCLC) and small cell lung cancer (SCLC). NSCLC is a heterogeneous disease that consists of adenocarcinoma, large cell carcinoma, and squamous cell carcinoma, and comprises approximately 84% of all lung cancers. The average age of diagnosis is approximately 70 years old. Despite advances in early detection and standard treatment, NSCLC is often diagnosed at an advanced stage, has poor prognosis, and is the leading cause of cancer deaths worldwide. In the US, the 5-year overall survival (OS) rate of all stages of NSCLC is approximately 19%, with advanced patients (Stage IIIB/IV) having a 5-year OS rate of <5%.

Platinum based doublet chemotherapy, maintenance chemotherapy, and anti-angiogenic agents in combination with chemotherapy have contributed to improved patient outcomes in advanced NSCLC. Until recently, first line platinum-based chemotherapy was the only standard treatment approach for most patients with NSCLC without targetable oncogene drivers. However, recent understanding of the interactions between the immune system and tumor growth has led to the development of a new class of immunotherapies, which have now moved to the frontline setting for NSCLC treatment. Despite this progress, there nevertheless is a need for additional therapies for the treatment of NSCLC.

### SUMMARY

In one aspect, the disclosure provides a method of treating non-small cell lung cancer (NSCLC) in a patient previously treated with a combination of (i) an immune checkpoint inhibitor that is not dostarlimab or a biosimilar thereof and (ii) one or more chemotherapeutic agents, the method comprising administering a therapeutically effective amount of dostarlimab or a biosimilar thereof to the patient, wherein the patient discontinued administration of the immune checkpoint inhibitor prior to beginning administration of dostarlimab or a biosimilar thereof.

In one aspect, the disclosure provides a method of treating non-small cell lung cancer (NSCLC) in a patient previously treated with a combination of pembrolizumab or a biosimilar thereof, and one or more chemotherapeutic agents, the method comprising administering a therapeutically effective amount of dostarlimab or a biosimilar thereof, and one or more chemotherapeutic agents to the patient.

In one aspect, the disclosure provides a method of treating non-small cell lung cancer (NSCLC) in a patient, the method comprising: (a) selecting a patient previously treated with a combination of (i) an immune checkpoint inhibitor that is not dostarlimab or a biosimilar thereof and (ii) one or more chemotherapeutic agents; and (b) administering a therapeutically effective amount of dostarlimab or a biosimilar thereof to the patient; wherein the patient discontinued administration of the immune checkpoint inhibitor prior to beginning administration of dostarlimab of a biosimilar thereof.

In one aspect, the disclosure provides a method of treating non-small cell lung cancer (NSCLC) in a patient, the method comprising: (a) selecting a patient previously treated with a combination of pembrolizumab or a biosimilar thereof, and one or more chemotherapeutic agents; and (b) administering a therapeutically effective amount of dostarlimab or a biosimilar thereof to the patient.

In one aspect, the disclosure provides the use of dostarlimab or a biosimilar thereof for the treatment of non-small cell lung cancer (NSCLC) in a patient previously treated with a combination of (i) an immune checkpoint inhibitor that is not dostarlimab or a biosimilar thereof and (ii) one or more chemotherapeutic agents.

In an embodiment, the immune checkpoint inhibitor is selected from the group consisting of a programmed cell death protein 1 (PD-1) antagonist, a programmed cell death ligand 1 (PD-L1) antagonist, a cytotoxic T-lymphocyte-associated protein 4 (CTLA-4) antagonist, and a lymphocyte-activation gene 3 (LAG-3) antagonist. In an embodiment, the PD-1 antagonist is an anti-PD-1 antibody. In an embodiment, the anti-PD-1 antibody is selected from the group consisting of pembrolizumab or a biosimilar thereof, nivolumab or a biosimilar thereof, and cemiplimab or a biosimilar thereof. In an embodiment, the anti-PD-1 antibody is pembrolizumab or a biosimilar thereof. In an embodiment, the PD-L1 antagonist is an anti-PD-L1 antibody. In an embodiment, the anti-PD-L1 antibody is selected from the group consisting of atezolizumab or a biosimilar thereof, avelumab or a biosimilar thereof, and durvalumab or a biosimilar thereof. In an embodiment, the CTLA-4 antagonist is an anti-CTLA-4 antibody. In an embodiment, the anti-CTLA-4 antibody is ipilimumab or a biosimilar thereof. In an embodiment, the LAG-3 antagonist is an anti-LAG-3 antibody. In an embodiment, the anti-LAG-3 antibody is relatlimab or a biosimilar thereof. In an embodiment, the anti-LAG-3 antibody is encelimab (also known as TSR-033) or a biosimilar thereof.

In an embodiment, the one or more chemotherapeutic agents are selected from the group consisting of an antifolate antineoplastic agent, a platinum-based chemotherapy, and a combination of an antifolate antineoplastic agent and a platinum-based chemotherapy. In an embodiment, the antifolate antineoplastic agent is pemetrexed. In an embodiment, the platinum-based chemotherapy is cisplatin or carboplatin.

In an embodiment, the patient discontinued administration of at least one of the one or more chemotherapeutic agents prior to beginning administration of dostarlimab or a biosimilar thereof.

In an embodiment, at least one of the one or more chemotherapeutic agents are co-administered with dostarlimab or a biosimilar thereof.

In an embodiment, the patient had progressive disease or stable disease prior to treatment with dostarlimab or a biosimilar thereof.

In an embodiment, the PD-1 antagonist is administered at a dose of 200 mg. In an embodiment, the PD-1 antagonist is administered through a 30 minute infusion Q3W. In an embodiment, the PD-1 antagonist is administered at a dose of 400 mg. In an embodiment, the PD-1 antagonist is administered through a 30 minute infusion Q6W. In an embodiment, the pemetrexed is administered at a dose of 500 milligram per meter square (mg/m²). In an embodiment, the pemetrexed is administered through a 10 minute IV infusion Q3W. In an embodiment, the cisplatin is administered at a dose of 75 mg/m². In an embodiment, the cisplatin is administered through a 30 minute IV infusion Q3W for 4 cycles. In an embodiment, the carboplatin is administered at a dose at an area under the concentration time curve of 5 milligram/milliliters/minute (mg/mL/min). In an embodiment, the carboplatin is administered through a 15 to 60 minute IV infusion Q3W for 4 cycles.

In an embodiment, the dostarlimab or a biosimilar thereof is administered at a dose of 500 mg. In an embodiment, the dostarlimab or a biosimilar thereof is administered through a 30 minute infusion intravenously Q3W. In an embodiment, the dostarlimab or a biosimilar thereof is administered at a dose of 1,000 mg beginning 3 weeks after dose 4. In an embodiment, the dostarlimab or a biosimilar thereof is administered every 6 weeks.

In an embodiment, administration of dostarlimab, or a biosimilar thereof, results in improved objective response rate (ORR) relative to administration of the immune checkpoint inhibitor that is not dostarlimab, or a biosimilar thereof, and the one or more chemotherapeutic agents. In an embodiment, the method demonstrates non-inferiority to administration of the immune checkpoint inhibitor that is not dostarlimab, or a biosimilar thereof, and the one or more chemotherapeutic agents. In an embodiment, non-inferiority is demonstrated using ORR. In an embodiment, the ORR is a proportion of participants with a best overall response of confirmed complete response or partial response in an analysis population. In an embodiment, non-inferiority is demonstrated using a process comprising monitoring release of one or more cytokines from PDE. In an embodiment, the one or more cytokines are selected from the group consisting of IL-2, IFN-γ, IP-10, and I-TAC.

In an embodiment, the NSCLC is metastatic NSCLC. In an embodiment, the NSCLC is locally advanced. In an embodiment the NSCLC is locally advanced, unresectable NSCLC.

In an embodiment, the NSCLC is a large cell neuroendocrine carcinoma (LCNEC), a lung adenocarcinoma (LUAD), or a lung squamous cell carcinoma (LUSC). In an embodiment, the NSCLC is of a subtype selected from acinar LUAD, LCNEC, micropapillary LUAD, mucinous LUAD, papillary LUAD, LUSC, or solid LUAD.

In an embodiment, the patient has a PD-L1 tumor proportion score (TPS) of 50% or greater.

It is understood that any and all embodiments of the present disclosure may be taken in combination with any other embodiment or embodiments to describe additional embodiments. It is also to be understood that each individual element of certain embodiments is its own independent embodiment. Furthermore, any element of an embodiment is meant to be combined with any and all other elements from any embodiment to describe an additional embodiment.

### BRIEF DESCRIPTION OF THE FIGURES

FIG. 1 is a graph depicting overall response rate (ORR) according to blinded independent central review (BICR) of dostarlimab plus chemotherapy compared to pembrolizumab plus chemotherapy.

FIG. 2 shows overall response rate (ORR) according to blinded independent central review (BICR) of dostarlimab plus chemotherapy compared to pembrolizumab plus chemotherapy, stratified by PD-L1 status.

FIG. 3 shows progression free survival (PFS) by RECIST v1.1 of dostarlimab plus chemotherapy compared to pembrolizumab plus chemotherapy.

FIG. 4 shows the safety results of dostarlimab plus chemotherapy compared to pembrolizumab plus chemotherapy.

FIG. 5 is a graph depicting tumor subtype distribution (absolute count) of the NSCLC PDE (patient derived explant) dataset.

FIG. 6 shows that NSCLC tumors show differential immune activation following dostarlimab treatment. FIG. 6 shows cytokine release in the NSCLC PDE model in response to dostarlimab treatment after 48 h in 54 donors, displayed as fold change normalized to the respective IgG control. Each bar represents the median fold change of n=4 biological replicates (each replicate consisting of n=4 PCS) per treatment condition.

FIG. 7 shows that response to dostarlimab is similar to the response to pembrolizumab in the NSCLC PDE model. FIG. 7 depicts graphs showing the fold changes of IFN-γ (left) and IL-2 (right) release in response to dostarlimab and pembrolizumab, normalized to IgG, from the PDE model for N=15 NSCLC donors. Each point corresponds to one PDE donor and represents the median fold change to IgG from 2 to 4 biological replicates. The solid line corresponds to the equality line, with the observations following the line suggesting a positive association.

FIG. 8 shows that there is no significant difference in IL-2 EC₅₀ or IFN-γ EC₅₀ between dostarlimab and pembrolizumab in the NSCLC PDE model. FIG. 8 depicts graphs showing induction of IL-2 (left) or IFN-γ (right) by dostarlimab and pembrolizumab in relation to the concentration applied in the PDE model from 12 individual NSCLC donors. The mean log 2-fold change (log 2FC) induction (dostarlimab (solid line with crosshatched circular markers) and pembrolizumab (dashed line with open circular markers), each vs. baseline IgG control (y-axis)) in relation to the applied antibody concentrations (x-axis) are displayed in Goodness-of-fit plots. Solid line and dashed lines denote the nonlinear mixed effects model prediction of cytokine induction for dostarlimab and pembrolizumab, respectively. Shaded dense crosshatching region and sparse cross hatching region denote the 90% confidence interval for cytokine induction by dostarlimab (dense crosshatching) and pembrolizumab (sparse crosshatching), respectively, generated from Monte Carlo simulations.

FIG. 9 depicts identification of NSCLC subgroups responding to dostarlimab. FIG. 9 is a scatter plot showing the induction of IFN-γ (y-axis) and IL-2 (x-axis) release in N=54 NSCLC donors in the PDE model treated with dostarlimab. The cytokine secretion by dostarlimab is displayed as fold change, normalized to isotope IgG. Each point corresponds to one PDE donor and represents the median fold change from 2 to 4 biological replicates. The solid line at x=2.71 and at y=2.84 indicates the cut-off employed to group donors into those showing a strong immune activation by dostarlimab (open circles) and those with weak or no immune activation (crosshatched circles). Next to the x- and y-axes, density distributions of the respective FC values are displayed.

FIG. 10 depicts increased immune activation by dostarlimab in donors with higher PD-L1 expression and CD8 infiltration. FIG. 10A depicts bar plots showing the fraction of responders (R) vs. non-responders (NR) to dostarlimab in the PDE model in the categories of PD-L1 TPS scores (left), PD-L1 IC-Score (middle) and CD8+ T cell-infiltration (right). CD8 high: ≥6.4%, CD8 low: <6.4% of all detected cells, N: number of NSCLC donors per category.

FIG. 10B shows representative IHC images of NSCLC tumors with high (left) or low (right) CD8+ T cell infiltration.

FIG. 11 depicts donor and tumor characteristics of responders (R) and non-responders (NR) to dostarlimab in the NSCLC PDE model. FIG. 11 shows bar plots showing the fraction of responders (R) vs. non-responders (NR) to dostarlimab in the NSCLC PDE model in the categories of main diagnosis, tumor stage, tumor grading, donor gender and smoking history. N: number of NSCLC donors per category.

FIG. 12 shows that immune activation is heterogeneous across different NSCLC subtypes. FIG. 12A depicts bar plots showing the fraction of responders (R) vs. non-responders (NR) to dostarlimab in the PDE model in different types of NSCLC growth patterns.

FIG. 12B shows representative images of NSCLC tumors with solid (left) and acinar (right) growth patterns (H&E stained). LUAD: lung adenocarcinoma, LCNEC: large cell neuroendocrine carcinoma, LUSC: lung squamous cell carcinoma.

FIG. 13 shows that baseline expression of CD137 in specific CD8+ and CD4+ T cell subtypes correlates with immune activation in response to dostarlimab treatment in the NSCLC PDE model. FIG. 13 depicts scatter plots showing the fold changes of IFN-γ release in response to dostarlimab (normalized to IgG and expressed as fold change, x axis) from the PDE model and the median fluorescence intensity (MFI, y axis) of CD137 in the total CD8+ T-cell population (left) as well as six T-cell subtypes (right), measured by flow cytometry at baseline for N=16 NSCLC donors. Each point corresponds to one NSCLC donor and represents the median fold change from n=2-4 (IFN-γ) or n=1 (CD137 MFI) biological replicates. The solid line corresponds to the linear mixed model fit, with the associated 95% confidence interval (shaded area) and p value: low p values suggest a significant linear association.

FIG. 14 shows a schematic of the antigen presenting cell (APC) PD-1 effector cell assay.

FIG. 15 shows dose response curves for various anti-PD-1 and anti-PD-L1 antibodies in the effector cell assay depicted in FIG. 14. FIG. 15A shows the fold-activation over the IgG4 isotype control for Keytruda, Opdivo, and dostarlimab for two independent replicates (N1 and N2) over an approximately 5-log concentration range. FIG. 15B shows the fold-activation over the IgG4 isotype control for Tecentriq, Bavencio, Imfinzi, Keytruda, Opdivo, and dostarlimab for two independent replicates (N1 and N2) over an approximately 5-log concentration range.

### DETAILED DESCRIPTION

### Definitions

As used herein and in the claims, the term “comprising” encompasses “Including” or “consisting,” e.g., a composition “comprising” X may consist exclusively of X or may include something additional, e.g., X+Y.

Unless defined otherwise, all technical and scientific terms used herein have the same meaning as commonly understood by one of ordinary skill in the art to which this disclosure pertains. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods and uses disclosed herein.

Concentrations described herein are determined at ambient temperature and pressure. This may be, for example, the temperature and pressure at room temperature or within a particular portion of a process stream. Preferably, concentrations are determined at a standard state of 25° C. and 1 bar of pressure.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long): an amino-terminal variable (VH) domain (located at the tips of the Y structure), followed by three constant domains: CH1, CH2, and the carboxy-terminal CH3 (located at the base of the Y's stem). A short region, known as the “switch,” connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains: an amino-terminal variable (VL) domain, followed by a carboxy-terminal constant (CL) domain, separated from one another by another “switch.” Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered glycosylation. For purposes of the present disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, fully human, and the like, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA. IgG, IgE or IgM antibodies; bi- or multi-specific antibodies; antibody fragments such as Fab fragments, Fab′ fragments, F(ab′)2 fragments, Fd′ fragments, Fd fragments, and isolated CDRs or sets thereof; single chain Fvs; polypeptide-Fc fusions; and single domain antibodies. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, and the like), or other pendant group (e.g., poly-ethylene glycol, and the like)).

As used herein, the term “complete response” or “CR” is used to mean the disappearance of all or substantially all target lesions. Any pathological lymph nodes must be <10 mm in the short axis. In some embodiments, CR refers to an about 80%, 85%, 90%, 91%, 92%, 93%, 94%, 95%, 96%, 97%, 98%, 99% or 100% decrease in the sum of the diameters of the target lesions (i.e., loss of lesions), taking as reference the baseline sum diameters. In some embodiments, CR indicates that less than about 10%, 9%, 8%, 7%, 6%, 5%, 4%, 3%, 2%, 1% or less of the total lesion diameter remains after treatment. Exemplary methods for evaluating complete response are identified by RECIST guidelines. See, e.g., E. A. Eisenhauer, et al., “New response evaluation criteria in solid tumors: Revised RECIST guideline (version 1.1.),” Eur. J. of Cancer, 45: 228-247 (2009).

As used herein, the term “partial response” or “PR” refers to a decrease in tumor progression in a subject as indicated by a decrease in the sum of the diameters of the target lesions, taking as reference the baseline sum diameters. In some embodiments, PR refers to at least a 30% decrease in the sum of diameters of target lesions, taking as reference the baseline sum diameters. Exemplary methods for evaluating partial response are identified by RECIST guidelines. See e.g., E. A. Eisenhauer, et al., “New response evaluation criteria in solid tumors: Revised RECIST guideline (version 1.1.),” Eur. J. of Cancer, 45: 228-247 (2009).

As used herein. “stabilization” of tumor growth or a “stable disease” (“SD”) refers to neither sufficient shrinkage to qualify for PR nor sufficient increase to qualify for PD. In some embodiments, stabilization refers to a less than 30%, 25%, 20%, 15%, 10% or 5% change (increase or decrease) in the sum of the diameters of the target lesions, taking as reference the baseline sum diameters. Exemplary methods for evaluating stabilization of tumor growth or a stable disease are identified by RECIST guidelines. See e.g., E. A. Eisenhauer, et al., “New response evaluation criteria in solid tumors: Revised RECIST guideline (version 1.1.),” Eur. J. of Cancer, 45: 228-247 (2009).

The term “progression” of tumor growth or a “progressive disease” (“PD”) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth.

The term “dostarlimab” refers to a programmed cell death protein 1 (PD-1)-blocking antibody (“anti-PD-1 antibody”), or a functional fragment or a functional variant thereof, comprising an immunoglobulin heavy chain variable domain (VH) comprising a complementary determining region (CDR) H1 comprising the amino acid sequence of SEQ ID NO: 21: a CDRH2 comprising the amino acid sequence of SEQ ID NO: 22; a CDRH3 comprising the amino acid sequence of SEQ ID NO: 23; and an immunoglobulin light chain immunoglobulin variable domain (VL) comprising a CDRL1 comprising the amino acid sequence of SEQ ID NO: 24; a CDRL2 comprising the amino acid sequence of SEQ ID NO: 25; and a CDRL3 comprising the amino acid sequence of SEQ ID NO: 26. In certain embodiments, the VH comprises an amino acid sequence of SEQ ID NO: 27. In other embodiments, the VL comprises an amino acid sequence of SEQ ID NO: 28. In certain other embodiments, dostarlimab comprises a VH comprising an amino acid sequence of SEQ ID NO: 27 and a VL comprising an amino acid sequence of SEQ ID NO: 28.

PD-1 is a cell surface receptor expressed on T cells that limits T-cell activation through binding to its ligands, programmed cell death-ligand 1 (PD-L1), and to a lesser extent, programmed cell death-ligand 2 (PD-L2). PD-1 also limits tyrosine kinase signaling from the T cell antigen receptor and co-stimulatory receptors. The PD-1/PD-L1 checkpoint serves as a negative regulator of T cell activity to help control local inflammatory responses and maintain self-tolerance. Upregulation of PD-1 ligands occurs in some tumors, and signaling through this pathway can contribute to inhibition of active T-cell immune surveillance of tumors. Dostarlimab blocks the interaction between PD-1 and its ligands PD-L1 and PD-L2, thereby removing PD-1 pathway-mediated inhibition of the immune response, including the anti-tumor immune response.

The term “biosimilar” refers to a biopharmaceutical or a biologic product that is highly similar to a reference biologic product (e.g., dostarlimab) notwithstanding minor differences in clinically inactive components, and for which there are no clinically meaningful differences between the biologic product and the reference product in terms of the safety, purity, and potency of the product (Section 351(i) of the Public Health Service Act (42 U.S.C. 262(i))).

Therefore, the term “biosimilar” refers to a biologic product that is highly similar to the reference product (e.g., dostarlimab) approved by a regulatory agency (e.g., the Federal Drug Administration (FDA) or the European Medicines Agency (EMA)) based on data from (a) analytical studies demonstrating that the biologic product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biologic product and the reference product in terms of the safety, purity, and potency of the product). In certain embodiments, the biosimilar product is an interchangeable product as determined by a regulatory agency (e.g., the FDA).

It will be generally understood that a biosimilar of dostarlimab will comprise the post-translational modifications that are essential for the function and efficacy of dostarlimab (as disclosed in International Publication No. WO 2021/126657). However, in certain embodiments, the biosimilar (e.g., a biosimilar of dostarlimab) may comprise one or more molecular differences, such as post-translational modifications, for example, although not limited to, glycosylation, oxidation, deamidation, and/or truncation which is/are different to the post-translational modifications of the reference medicinal product (e.g., dostarlimab), provided that the differences do not result in a significant change in safety and/or efficacy of the medicinal product. In certain embodiments, the biosimilar (e.g., a biosimilar of dostarlimab) may have an identical or different glycosylation pattern to the reference medicinal product (e.g., dostarlimab). Particularly, although not exclusively, the biosimilar (e.g., a biosimilar of dostarlimab) may have a different glycosylation pattern, such as, if the differences address or are intended to address safety concerns associated with the reference medicinal product (e.g., dostarlimab). Additionally, the biosimilar (biosimilar (e.g., a biosimilar of dostarlimab) may deviate from the reference medicinal product (e.g., dostarlimab) in, for example, its strength, pharmaceutical form, formulation, excipients and/or presentation, providing safety and efficacy of the medicinal product is not compromised. In other embodiments, the biosimilar (e.g., a biosimilar of dostarlimab) may comprise differences in, for example, pharmacokinetic (PK) and/or pharmacodynamic (PD) profiles as compared to the reference medicinal product (e.g., dostarlimab) but is still deemed sufficiently similar to the reference medicinal product as to be authorized or considered suitable for authorization. In certain embodiments, the biosimilar (e.g., a biosimilar of dostarlimab) may exhibit different binding characteristics as compared to the reference medicinal product (e.g., dostarlimab), wherein the different binding characteristics are considered by a Regulatory Authority such as the FDA and/or EMA not to be a barrier for authorization as a similar biologic product. The term “biosimilar” is also used synonymously by other national and regional regulatory agencies.

It will be understood that a drug's international nonproprietary name (INN), (e.g., dostarlimab, pembrolizumab, nivolumab), is to be interpreted as including generic, bioequivalent, follow-on biologic, and/or biosimilar versions of that drug, including but not limited to any drug that has received abbreviated regulatory approval by reference to an earlier regulatory approval of that drug. Additionally, a drug's INN optionally includes, but is not limited to, glycosylation variants of dostarlimab, and biosimilars thereof.

### Methods of Treating Non-Small Cell Lung Cancer (NSCLC)

Described herein are methods of treating NSCLC in a patient that benefits from administration of immune checkpoint inhibitor therapy. Also described are uses of immune checkpoint inhibitor therapy in the treatment of NSCLC in a patient. This disclosure recognizes that switching patients from a previous immune checkpoint inhibitor therapy to therapy with dostarlimab or a biosimilar thereof benefits NSCLC patients. For example, after discontinuing the prior immune checkpoint inhibitor therapy, dostarlimab or a biosimilar is administered e.g., as a monotherapy or in combination therapy, for a period sufficient to achieve clinical benefit or according to a regimen as determined by a physician (e.g., dostarlimab or a biosimilar is administered in dosage amounts and number of treatment cycles as determined by a physician). In an embodiment, the patient had progressive disease prior to treatment with dostarlimab or a biosimilar thereof. In an embodiment, the patient had stable disease prior to treatment with dostarlimab or a biosimilar thereof. In an embodiment, the patient had progressive disease or stable disease prior to treatment with dostarlimab or a biosimilar thereof.

In one aspect, the disclosure provides methods of treating NSCLC in a patient by administering a therapeutically effective amount of dostarlimab or a biosimilar thereof to the patient. In an embodiment, the patient is a patient previously treated with a combination of (i) an immune checkpoint inhibitor that is not dostarlimab or a biosimilar thereof and (ii) one or more chemotherapeutic agents. In an embodiment, the patient discontinued administration of the immune checkpoint inhibitor that is not dostarlimab or a biosimilar thereof prior to beginning administration of dostarlimab or a biosimilar thereof.

In one aspect, the disclosure provides methods of treating NSCLC in a patient previously treated with a combination of pembrolizumab or a biosimilar thereof, and one or more chemotherapeutic agents, the method comprising administering a therapeutically effective amount of dostarlimab or a biosimilar thereof, and one or more chemotherapeutic agents to the patient. In an embodiment, the pembrolizumab or a biosimilar thereof is administered subcutaneously and the dostarlimab or a biosimilar thereof is administered intravenously.

In one aspect, the disclosure provides methods of treating NSCLC in a patient by (a) selecting a patient previously treated with a combination of (i) an immune checkpoint inhibitor that is not dostarlimab or a biosimilar thereof and (ii) one or more chemotherapeutic agents; and (b) administering a therapeutically effective amount of dostarlimab or a biosimilar thereof to the patient. In an embodiment, the patient discontinued administration of the immune checkpoint inhibitor that is not dostarlimab or a biosimilar thereof prior to beginning administration of dostarlimab of a biosimilar thereof.

In an embodiment, the disclosure provides methods of treating NSCLC in a patient, by (a) selecting a patient previously treated with a combination of pembrolizumab or a biosimilar thereof, and one or more chemotherapeutic agents; and (b) administering a therapeutically effective amount of dostarlimab or a biosimilar thereof to the patient. In an embodiment, the pembrolizumab or a biosimilar thereof is administered subcutaneously and the dostarlimab or a biosimilar thereof is administered intravenously.

In an embodiment, the disclosure provides methods of treating NSCLC in a patient, by (a) selecting a patient previously treated with a combination of nivolumab or a biosimilar thereof, and one or more chemotherapeutic agents; and (b) administering a therapeutically effective amount of dostarlimab or a biosimilar thereof to the patient. In an embodiment, the nivolumab or a biosimilar thereof is administered subcutaneously and the dostarlimab or a biosimilar thereof is administered intravenously.

In an embodiment, the disclosure provides methods of treating NSCLC in a patient, by (a) selecting a patient previously treated with an anti-PD-L1 antibody selected from the group consisting of atezolizumab, avelumab, durvalumab, and biosimilars thereof, and one or more chemotherapeutic agents; and (b) administering a therapeutically effective amount of dostarlimab or a biosimilar thereof to the patient.

In an embodiment, the disclosure provides methods of treating locally advanced NSCLC in a patient, by (a) selecting a patient previously treated with an anti-PD-L1 antibody selected from the group consisting of atezolizumab, avelumab, durvalumab, and biosimilars thereof, and one or more chemotherapeutic agents; and (b) administering a therapeutically effective amount of dostarlimab or a biosimilar thereof to the patient. In some embodiments, the patient has progressed following the previous treatment with an anti-PD-L1 antibody.

In an embodiment, the disclosure provides methods of treating NSCLC in a patient (a) wherein the NSCLC is unresectable, Stage III NSCLC; (b) wherein the NSCLC has not progressed following platinum-based chemotherapy and radiation therapy; and (c) administering a therapeutically effective amount of dostarlimab or a biosimilar thereof to the patient.

In an embodiment, the disclosure provides a method of treating non-small cell lung cancer (NSCLC) in a patient previously treated with chemotherapy and radiotherapy comprising administering dostarlimab or a biosimilar thereof 500 mg Q3W. In some embodiments, the chemotherapy is platinum-based chemotherapy. In some embodiments, the NSCLC is locally advanced.

In an embodiment, the disclosure provides the use of dostarlimab or a biosimilar thereof for the treatment of NSCLC in a patient previously treated with a combination of (i) an immune checkpoint inhibitor that is not dostarlimab or a biosimilar thereof and (ii) one or more chemotherapeutic agents. In an embodiment, the disclosure provides the use of dostarlimab or a biosimilar thereof for the treatment of NSCLC in a patient previously treated with a combination of (i) pembrolizumab or a biosimilar thereof and (ii) one or more chemotherapeutic agents.

In an embodiment, the NSCLC is metastatic NSCLC. In an embodiment, the NSCLC is non-squamous NSCLC. In an embodiment, the NSCLC is metastatic non-squamous NSCLC. In an embodiment, the NSCLC is NSCLC without a known epidermal growth factor receptor (EGFR), anaplastic lymphoma kinase (ALK), receptor tyrosine kinase-1 (ROS-1), or proto-oncogene B-raf (BRAF) V600E mutation. In an embodiment, the NSCLC is NSCLC without a genomic aberration for which an approved targeted therapy is available. In an embodiment, the NSCLC is NSCLC without a known EGFR, ALK, ROS-1, or BRAF V600E mutation or other genomic aberration for which an approved targeted therapy is available. In an embodiment, the NSCLC is metastatic NSCLC without a known EGFR, ALK, ROS-1, or BRAF V600E mutation. In an embodiment, the NSCLC is metastatic NSCLC without a known EGFR, ALK, ROS-1, or BRAF V600E mutation or other genomic aberration for which an approved targeted therapy is available. In an embodiment, the NSCLC is non-squamous NSCLC without a known EGFR, ALK, ROS-1, or BRAF V600E mutation. In an embodiment, the NSCLC is non-squamous NSCLC without a known EGFR, ALK, ROS-1, or BRAF V600E mutation or other genomic aberration for which an approved targeted therapy is available. In an embodiment, the NSCLC is metastatic non-squamous NSCLC without a known EGFR, ALK, ROS-1, or BRAF V600E mutation. In an embodiment, the NSCLC is metastatic non-squamous NSCLC without a known EGFR, ALK, ROS-1, or BRAF V600E mutation or other genomic aberration for which an approved targeted therapy is available.

In an embodiment, the NSCLC is a large cell neuroendocrine carcinoma (LCNEC), a lung adenocarcinoma (LUAD), or a lung squamous cell carcinoma (LUSC). In an embodiment, the NSCLC is a lung adenocarcinoma (LUAD) or a lung squamous cell carcinoma (LUSC). In an embodiment, the NSCLC is of a subtype selected from acinar LUAD, LCNEC, micropapillary LUAD, mucinous LUAD, papillary LUAD, LUSC, or solid LUAD. In an embodiment, the NSCLC is of a subtype selected from acinar LUAD, micropapillary LUAD, LUSC, or solid LUAD. In an embodiment, the NSCLC is of a subtype selected from acinar LUAD, LUSC, or solid LUAD. In an embodiment, the NSCLC is of a subtype selected from LUSC or solid LUAD. In an embodiment, the NSCLC is of a subtype that is acinar LUAD. In an embodiment, the NSCLC is of a subtype that is LUSC. In an embodiment, the NSCLC is of a subtype that is solid LUAD.

In an embodiment, the patient has a PD-L1 TPS of 1% or greater. In an embodiment, the patient has a PD-L1 TPS of 10% or greater. In an embodiment, the patient has a PD-L1 TPS of 25% or greater. In an embodiment, the patient has a PD-L1 TPS of 50% or greater. In an embodiment, the patient has a PD-L1 immune cell (IC) score of 10% or greater.

### Immune Checkpoint Inhibitors

The present disclosure is directed to methods of treating and uses for treatment of NSCLC in a patient previously treated with an immune checkpoint inhibitor that is not dostarlimab or a biosimilar thereof. In an embodiment, the immune checkpoint inhibitor is selected from the group consisting of a programmed cell death protein 1 (PD-1) antagonist, a programmed cell death ligand 1 (PD-L1) antagonist, a cytotoxic T-lymphocyte-associated protein 4 (CTLA-4) antagonist, and a lymphocyte-activation gene 3 (LAG-3) antagonist. The immune checkpoint inhibitors of the disclosure include biosimilars thereof.

As used herein, “PD-1 antagonist” means any chemical compound or biological molecule that blocks binding of PD-ligand 1 (PD-L1) expressed on a cancer cell to PD-1 expressed on an immune cell (T cell, B cell or natural killer T (NKT) cell) and preferably also blocks binding of PD ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in certain embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the PD-1 antagonist is an anti-PD-1 antibody. In an embodiment, the anti-PD-1 antibody is selected from the group consisting of pembrolizumab or a biosimilar thereof, nivolumab or a biosimilar thereof, and cemiplimab or a biosimilar thereof. In an embodiment, the anti-PD-1 antibody is pembrolizumab or a biosimilar thereof.

Pembrolizumab is a humanized monoclonal anti-PD-1 antibody commercially available as KEYTRUDA. Pembrolizumab is indicated for the treatment of some unresectable or metastatic melanomas. The amino acid sequence of pembrolizumab and methods of using are disclosed in U.S. Pat. No. 8,168,757.

Nivolumab is a humanized monoclonal anti-PD-1 antibody commercially available as OPDIVO. Nivolumab is indicated for the treatment of some unresectable or metastatic melanomas. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Cemiplimab is a human monoclonal anti-PD-1 antibody commercially available as LIBTAYO. Cemiplimab is indicated for the treatment of non-small cell lung cancer (NSCLC), basal cell carcinoma (BCC), and cutaneous squamous cell carcinoma (CSCC).

As used herein, “-PD-L1 antagonist” means any chemical compound or biological molecule that blocks binding of PD-ligand 1 (PD-L1) to one or more of its binding partners, such as PD-1 or B7-1. PD-L1 antagonists include a monoclonal antibody (mAb), or antigen binding fragment thereof, which specifically binds to PD-L1, and preferably specifically binds to human PD-LL. The mAb may be a human antibody, a humanized antibody or a chimeric antibody, and may include a human constant region. In some embodiments, the human constant region is selected from the group consisting of IgG1, IgG2, IgG3 and IgG4 constant regions, and in certain embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the PD-L1 antagonist is an anti-PD-L1 antibody. In an embodiment, the anti-PD-L1 antibody is selected from the group consisting of atezolizumab or a biosimilar thereof, avelumab or a biosimilar thereof, and durvalumab or a biosimilar thereof.

Atezolizumab is a fully humanized monoclonal anti-PD-L1 antibody commercially available as TECENTRIQ. Atezolizumab is indicated for the treatment of some locally advanced or metastatic urothelial carcinomas. Atezolizumab blocks the interaction of PD-L1 with PD-1 and CD80. Other exemplary PD-L1 antibodies include avelumab (BAVENCIO), durvalumab (IMFINZI).

As used herein, “CTLA-4 antagonist” means any chemical compound or biological molecule that blocks binding of CTLA-4 to one or more of its binding partners. CTLA-4 antagonists include a monoclonal antibody (mAb), or antigen binding fragment thereof, which specifically binds to CTLA-4, and preferably specifically binds to human CTLA-4. The mAb may be a human antibody, a humanized antibody or a chimeric antibody, and may include a human constant region. In some embodiments, the human constant region is selected from the group consisting of IgG1, IgG2, IgG3 and IgG4 constant regions, and in certain embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the CTLA-4 antagonist is an anti-CTLA-4 antibody. In an embodiment, the anti-CTLA-4 antibody is ipilimumab or a biosimilar thereof.

As used herein, “LAG-3 antagonist” means any chemical compound or biological molecule that blocks binding of LAG-3 to one or more of its binding partners. LAG-3 antagonists include a monoclonal antibody (mAb), or antigen binding fragment thereof, which specifically binds to LAG-3, and preferably specifically binds to human LAG-3. The mAb may be a human antibody, a humanized antibody or a chimeric antibody, and may include a human constant region. In some embodiments, the human constant region is selected from the group consisting of IgG1, IgG2, IgG3 and IgG4 constant regions, and in certain embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the LAG-3 antagonist is an anti-LAG-3 antibody. In an embodiment, the anti-LAG-3 antibody is relatlimab or a biosimilar thereof. In an embodiment, the anti-LAG-3 antibody is encelimab (also known as TSR-033) or a biosimilar thereof.

Exemplary antibody heavy chain and light chain sequences are provided in Table 1. In an embodiment, dostarlimab comprises the heavy and light chain sequences of SEQ ID NO: 1 and SEQ ID NO: 2, respectively. In an embodiment, the immune checkpoint inhibitor comprises the heavy chain and light chain sequences of SEQ ID NO: 3 and SEQ ID NO: 4, respectively. In an embodiment, the immune checkpoint inhibitor comprises the heavy chain and light chain sequences of SEQ ID NO: 5 and SEQ ID NO: 6, respectively. In an embodiment, the immune checkpoint inhibitor comprises the heavy chain and light chain sequences of SEQ ID NO: 7 and SEQ ID NO: 8, respectively. In an embodiment, the immune checkpoint inhibitor comprises the heavy chain and light chain sequences of SEQ ID NO: 9 and SEQ ID NO: 10, respectively. In an embodiment, the immune checkpoint inhibitor comprises the heavy chain and light chain sequences of SEQ ID NO: 11 and SEQ ID NO: 12, respectively. In an embodiment, the immune checkpoint inhibitor comprises the heavy chain and light chain sequences of SEQ ID NO: 13 and SEQ ID NO: 14, respectively. In an embodiment, the immune checkpoint inhibitor comprises the heavy chain and light chain sequences of SEQ ID NO: 15 and SEQ ID NO: 16, respectively. In an embodiment, the immune checkpoint inhibitor comprises the heavy chain and light chain sequences of SEQ ID NO: 17 and SEQ ID NO: 18, respectively.

TABLE 1

| SEQ ID | | |
|---|---|---|
| NO | Name | Sequence |
| 1 | Dostarlimab | EVQLLESGGGLVQPGGSLRLSCAASGFTFSSYDMSWVRQ |
| | Heavy Chain | APGKGLEWVSTISGGGSYTYYQDSVKGRFTISRDNSKNT |
| | | LYLQMNSLRAEDTAVYYCASPYYAMDYWGQGTTVTVS |
| | | SASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHN |
| | | HYTQKSLSLSLGK |
| 2 | Dostarlimab | DIQLTQSPSFLSAYVGDRVTITCKASQDVGTAVAWYQQK |
| | Light Chain | PGKAPKLLIYWASTLHTGVPSRFSGSGSGTEFTLTISSLQP |
| | | EDFATYYCQHYSSYPWTFGQGTKLEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 3 | Pembrolizumab | QVQLVQSGVEVKKPGASVKVSCKASGYTFTNYYMYWV |
| | Heavy Chain | RQAPGQGLEWMGGINPSNGGTNFNEKFKNRVTLTTDSST |
| | | TTAYMELKSLQFDDTAVYYCARRDYRFDMGFDYWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSS |
| | | SLGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTLP |
| | | PSQEEMTKNQVSLTCLVKGFYPSDIAVEWESNGQPENNY |
| | | KTTPPVLDSDGSFFLYSRLTVDKSRWQEGNVFSCSVMHE |
| | | ALHNHYTQKSLSLSLGK |
| 4 | Pembrolizumab | EIVLTQSPATLSLSPGERATLSCRASKGVSTSGYSYLHWY |
| | Light Chain | QQKPGQAPRLLIYLASYLESGVPARFSGSGSGTDFTLTISS |
| | | LEPEDFAVYYCQHSRDLPLTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 5 | Nivolumab | QVQLVESGGGVVQPGRSLRLDCKASGITFSNSGMHWVR |
| | Heavy Chain | QAPGKGLEWVAVIWYDGSKRYYADSVKGRFTISRDNSK |
| | | NTLFLQMNSLRAEDTAVYYCATNDDYWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSVF |
| | | LFPPKPKDTLMISRTPEVTCVVVDVSQEDPEVQFNWYVD |
| | | GVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPV |
| | | LDSDGSFFLYSRLTVDKSRWQEGNVFSCSVMHEALHNH |
| | | YTQKSLSLSLGK |
| 6 | Nivolumab | EIVLTQSPATLSLSPGERATLSCRASQSVSSYLAWYQQKP |
| | Light Chain | GQAPRLLIYDASNRATGIPARFSGSGSGTDFTLTISSLEPED |
| | | FAVYYCQQSSNWPRTFGQGTKVEIKRTVAAPSVFIFPPSD |
| | | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| 7 | Cemiplimab | EVQLLESGGVLVQPGGSLRLSCAASGFTFSNFGMTWVRQ |
| | Heavy Chain | APGKGLEWVSGISGGGRDTYFADSVKGRFTISRDNSKNT |
| | | LYLQMNSLKGEDTAVYYCVKWGNIYFDYWGQGTLVTV |
| | | SSASTKGPSVFPLAPCSRSTSESTAALGCLVKDYFPEPVTV |
| | | SWNSGALTSGVHTFPAVLQSSGLYSLSSVVTVPSSSLGTK |
| | | TYTCNVDHKPSNTKVDKRVESKYGPPCPPCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHN |
| | | HYTQKSLSLSLGK |
| 8 | Cemiplimab | DIQMTQSPSSLSASVGDSITITCRASLSINTFLNWYQQKPG |
| | Light Chain | KAPNLLIYAASSLHGGVPSRFSGSGSGTDFTLTIRTLQPED |
| | | FATYYCQQSSNTPFTFGPGTVVDFRRTVAAPSVFIFPPSDE |
| | | QLKSGTASVVCLLNNFYPREAKVQWKVDNALQSGNSQE |
| | | SVTEQDSKDSTYSLSSTLTLSKADYEKHKVYACEVTHQG |
| | | LSSPVTKSFNRGEC |
| 9 | Atezolizumab | EVQLVESGGGLVQPGGSLRLSCAASGFTFSDSWIHWVRQ |
| | Heavy Chain | APGKGLEWVAWISPYGGSTYYADSVKGRFTISADTSKNT |
| | | AYLQMNSLRAEDTAVYYCARRHWPGGFDYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVK |
| | | FNWYVDGVEVHNAKTKPREEQYASTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 10 | Atezolizumab | DIQMTQSPSSLSASVGDRVTITCRASQDVSTAVAWYQQK |
| | Light Chain | PGKAPKLLIYSASFLYSGVPSRFSGSGSGTDFTLTISSLQPE |
| | | DFATYYCQQYLYHPATFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 11 | Avelumab | EVQLLESGGGLVQPGGSLRLSCAASGFTFSSYIMMWVRQ |
| | Heavy Chain | APGKGLEWVSSIYPSGGITFYADTVKGRFTISRDNSKNTL |
| | | YLQMNSLRAEDTAVYYCARIKLGTVTTVDYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 12 | Avelumab Light | QSALTQPASVSGSPGQSITISCTGTSSDVGGYNYVSWYQQ |
| | Chain | HPGKAPKLMIYDVSNRPSGVSNRFSGSKSGNTASLTISGL |
| | | QAEDEADYYCSSYTSSSTRVFGTGTKVTVLGQPKANPTV |
| | | TLFPPSSEELQANKATLVCLISDFYPGAVTVAWKADGSPV |
| | | KAGVETTKPSKQSNNKYAASSYLSLTPEQWKSHRSYSCQ |
| | | VTHEGSTVEKTVAPTECS |
| 13 | Durvalumab | EVQLVESGGGLVQPGGSLRLSCAASGFTFSRYWMSWVR |
| | Heavy Chain | QAPGKGLEWVANIKQDGSEKYYVDSVKGRFTISRDNAK |
| | | NSLYLQMNSLRAEDTAVYYCAREGGWFGELAFDYWGQ |
| | | GTLVTVSSASTKGPSVFPLAPSSKSTSGGTAALGCLVKDY |
| | | FPEPVTVSWNSGALTSGVHTFPAVLQSSGLYSLSSVVTVP |
| | | SSSLGTQTYICNVNHKPSNTKVDKRVEPKSCDKTHTCPPC |
| | | PAPEFEGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPASIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| 14 | Durvalumab | EIVLTQSPGTLSLSPGERATLSCRASQRVSSSYLAWYQQK |
| | Light Chain | PGQAPRLLIYDASSRATGIPDRFSGSGSGTDFTLTISRLEPE |
| | | DFAVYYCQQYGSLPWTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 15 | Ipilimumab | QVQLVESGGGVVQPGRSLRLSCAASGFTFSSYTMHWVR |
| | Heavy Chain | QAPGKGLEWVTFISYDGNNKYYADSVKGRFTISRDNSKN |
| | | TLYLQMNSLRAEDTAIYYCARTGWLGPFDYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKRVEPKSCDKTHTCPPCPAPEL |
| | | LGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 16 | Ipilimumab | EIVLTQSPGTLSLSPGERATLSCRASQSVGSSYLAWYQQK |
| | Light Chain | PGQAPRLLIYGAFSRATGIPDRFSGSGSGTDFTLTISRLEPE |
| | | DFAVYYCQQYGSSPWTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 17 | Relatlimab | QVQLQQWGAGLLKPSETLSLTCAVYGGSFSDYYWNWIR |
| | Heavy Chain | QPPGKGLEWIGEINHRGSTNSNPSLKSRVTLSLDTSKNQF |
| | | SLKLRSVTAADTAVYYCAFGYSDYEYNWFDPWGQGTLV |
| | | TVSSASTKGPSVFPLAPCSRSTSESTAALGCLVKDYFPEPV |
| | | TVSWNSGALTSGVHTFPAVLQSSGLYSLSSVVTVPSSSLG |
| | | TKTYTCNVDHKPSNTKVDKRVESKYGPPCPPCPAPEFLG |
| | | GPSVFLFPPKPKDTLMISRTPEVTCVVVDVSQEDPEVQFN |
| | | WYVDGVEVHNAKTKPREEQFNSTYRVVSVLTVLHQDW |
| | | LNGKEYKCKVSNKGLPSSIEKTISKAKGQPREPQVYTLPP |
| | | SQEEMTKNQVSLTCLVKGFYPSDIAVEWESNGQPENNYK |
| | | TTPPVLDSDGSFFLYSRLTVDKSRWQEGNVFSCSVMHEA |
| | | LHNHYTQKSLSLSLG |
| 18 | Relatlimab | EIVLTQSPATLSLSPGERATLSCRASQSISSYLAWYQQKPG |
| | Light Chain | QAPRLLIYDASNRATGIPARFSGSGSGTDFTLTISSLEPEDF |
| | | AVYYCQQRSNWPLTFGQGTNLEIKRTVAAPSVFIFPPSDE |
| | | QLKSGTASVVCLLNNFYPREAKVQWKVDNALQSGNSQE |
| | | SVTEQDSKDSTYSLSSTLTLSKADYEKHKVYACEVTHQG |
| | | LSSPVTKSFNRGEC |
| 19 | Encelimab | EVQLVQSGAEVKKPGATVKISCKASGFSIKDDYIHWVQQ |
| | Heavy Chain | APGKGLEWMGWIDAMNDDSQYSSKFQGRVTITVDTSTN |
| | | TAYMKLSSLRSEDTAVYYCTYAFGGYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSVF |
| | | LFPPKPKDTLMISRTPEVTCVVVDVSQEDPEVQFNWYVD |
| | | GVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPV |
| | | LDSDGSFFLYSRLTVDKSRWQEGNVFSCSVMHEALHNH |
| | | YTQKSLSLSLGK |
| 20 | Encelimab | DIVMTQTPLSLSVTPGQPASISCRSSQSLVHSDSNTYLHW |
| | Light Chain | YLQKPGQSPQLLIYLVSNRFSGVPDRFSGSGSGTDFTLKIS |
| | | RVEAEDVGVYFCGQSTHVPYAFGGGTKVEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYA |
| | | CEVTHQGLSSPVTKSFNRGEC |
| 21 | Dostarlimab | SYDMS |
| | CDRH1 | |
| 22 | Dostarlimab | TISGGGSYTYYQDSVKG |
| | CDRH2 | |
| 23 | Dostarlimab | PYYAMDY |
| | CDRH3 | |
| 24 | Dostarlimab | KASQDVGTAVA |
| | CDHL1 | |
| 25 | Dostarlimab | WASTLHT |
| | CDRL2 | |
| 26 | Dostarlimab | QHYSSYPWT |
| | CDRL3 | |
| 27 | Dostarlimab | EVQLLESGGGLVQPGGSLRLSCAASGFTFSSYDMSWVRQ |
| | VH | APGKGLEWVSTISGGGSYTYYQDSVKGRFTISRDNSKNT |
| | | LYLQMNSLRAEDTAVYYCASPYYAMDYWGQGTTVTVS |
| | | S |
| 28 | Dostarlimab | DIQLTQSPSFLSAYVGDRVTITCKASQDVGTAVAWYQQK |
| | VL | PGKAPKLLIYWASTLHTGVPSRFSGSGSGTEFTLTISSLQP |
| | | EDFATYYCQHYSSYPWTFGQGTKLEIK |

### Biosimilars

In some embodiments, the biosimilar biologic product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biologic product have been previously approved for the reference product. In some embodiments, the route of administration, the dosage form, and/or the strength of the biologic product are the same as those of the reference product. A “biosimilar” can be, e.g., a presently known antibody having the same or similar primary amino acid sequence as a marketed antibody (e.g., dostarlimab), but may be made in different cell types or by different production, purification, or formulation methods, or in a different formulation. In some embodiments the biosimilar biologic product is a biosimilar of dostarlimab. In some embodiments the biosimilar biologic product is a biosimilar of pembrolizumab. In some embodiments the biosimilar biologic product is a biosimilar of nivolumab.

In certain embodiments of the methods described herein, dostarlimab can be replaced with a biosimilar thereof. Accordingly, it will be understood that the term “dostarlimab” is intended to encompass any biosimilar(s) of dostarlimab. Also encompassed by the term “dostarlimab” are antibodies which have CDRs, variable regions, and/or heavy and light chains of dostarlimab. Additionally encompassed by the term “dostarlimab” are antibodies and/or any biosimilar(s) of dostarlimab which have CDRs, variable regions, and/or heavy and light chains that are at least 70%, at least 75%, at least 80%, at least 85%, at least 90%, at least 95%, at least 96%, at least 97%, at least 98%, or at least 99% identical to the CDRs, variable regions, or heavy and light chains, respectively, of dostarlimab.

In certain embodiments, encompassed by the term “dostarlimab” are antibodies and/or any biosimilar(s) of dostarlimab which comprise heavy chain and light chain sequences that are identical to those in the reference antibody (i.e., dostarlimab), except for having at least six, five, four, three, two, or one amino acid substitutions at positions that are located within and/or outside of the light chain CDRs and/or at least six, five, four, three, two, or one conservative amino acid substitutions that are located within and/or outside of the heavy chain CDRs (e.g., the variant positions are located in the framework regions or the constant region), and wherein the antibody or biosimilar is substantially the same as the reference antibody (i.e., dostarlimab) with respect to the following properties: binding affinity to the inhibitory checkpoint molecule (i.e., PD-1) and ability to block the binding of the inhibitory checkpoint molecule (i.e., PD-1) to its ligand (i.e., PD-L1 and/or PD-L2).

In certain embodiments, dostarlimab is a PD-1-blocking humanized antibody. In other embodiments, dostarlimab is a PD-1-blocking monoclonal antibody (mAb). In yet other embodiments, dostarlimab is a PD-1-blocking humanized mAb of the Ig4 kappa isotype. In specific embodiments, dostarlimab comprises a heavy chain comprising the amino acid sequence of SEQ ID NO:1, and a light chain comprising the amino acid sequence of SEQ ID NO:2. In certain aspects, dostarlimab comprises of two heavy and two light chains with a single N-linked glycosylation site on each heavy chain (“dostarlimab-gxly”). In certain embodiments, the single glycosylation site is at asparagine 293 on the heavy chain. In certain embodiments, dostarlimab comprises a heavy chain comprising the amino acid sequence of SEQ ID NO:1, and a light chain comprising the amino acid sequence of SEQ ID NO:2, comprising all functional post-translational modifications thereof. In some embodiments, dostarlimab has a calculated molecular weight of about 144 kDa.

The primary structure and function of dostarlimab are disclosed in International Publication Nos. WO 2014/179664, WO 2018/085468 and WO 2018/129559, each of which is incorporated by reference herein in its entirety.

### Chemotherapy

The immune checkpoint inhibitors disclosed herein may be administered in combination with one or more chemotherapeutic agents. Suitable chemotherapeutic agents include antimetabolite neoplastic agents (e.g., antifolate antineoplastic agents), platinum-based chemotherapy, and other known chemotherapeutic agents.

Antimetabolite neoplastic agents are phase-specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, capecitabine, cladribine, clofarabine, cytarabine, decitabine, floxuridine, fludarabine, fluorouracil, gemcitabine, hydroxyurea, mercaptopurine, methotrexate, nelarabine, pemetrexed, pralatrexate, and thioguanine.

Platinum-based chemotherapeutic agents induce cancer cell apoptosis as a response to their covalent binding to DNA. Examples of platinum-based agents include, but are not limited to, cisplatin, carboplatin, oxaliplatin, nedaplatin, triplatin tetranitrate, phenanthriplatin, picoplatin, and satraplatin.

In an embodiment, the one or more chemotherapeutic agents are selected from the group consisting of an antifolate antineoplastic agent, a platinum-based chemotherapy, and a combination of an antifolate antineoplastic agent and a platinum-based chemotherapy.

In an embodiment, the antifolate antineoplastic agent is pemetrexed.

In an embodiment, the platinum-based chemotherapy is cisplatin. In an embodiment, the platinum-based chemotherapy is carboplatin.

In an embodiment, the platinum-based chemotherapy is platinum-doublet chemotherapy. In an embodiment the platinum-doublet chemotherapy is carboplatin AUC 5 or AUC 6 and, paclitaxel 175 mg/m² or 200 mg/m². In an embodiment the platinum-doublet chemotherapy is cisplatin 75 mg/m² and, pemetrexed 500 mg/m². In an embodiment the platinum-doublet chemotherapy is cisplatin 75 mg/m² day 1, gemcitabine 1000 mg/m² or 1250 mg/m² days. In an embodiment the platinum-doublet chemotherapy is cisplatin 75 mg/m² day, paclitaxel 175 mg/m² or 200 mg/m².

### Doses and Dosing Regimens

### Immune Checkpoint Inhibitors and Chemotherapeutic Agents

In some embodiments, the dose of the immune checkpoint inhibitor (e.g., an anti-PD-1 antibody such as pembrolizumab or a biosimilar thereof) is within a range of about 5 to about 5000 mg (e.g., about 5 mg, about 10 mg, about 50 mg, about 100 mg, about 200 mg, about 284 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, about 5000 mg, or a range defined by any two of the foregoing values). In some embodiments, the dose of the immune checkpoint inhibitor (e.g., an anti-PD-1 antibody such as pembrolizumab or a biosimilar thereof) is 200 mg, 284 mg, 500 mg, or 1000 mg.

In some embodiments, the dose is in an amount relative to body weight. In some embodiments, the dose of the immune checkpoint inhibitor (e.g., an anti-PD-1 antibody such as pembrolizumab) is within a range of about 0.01 mg/kg to 100 mg/kg of animal or human body weight; however, doses below or above this exemplary range are within the scope of the disclosure. The daily dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In some embodiments, the dose is administered according to a regimen that delivers a dose every two weeks. In some embodiments, the dose is administered according to a regimen that delivers a dose every three weeks. In some embodiments, the dose is administered according to a regimen that delivers a dose every four weeks. In some embodiments, the dose is administered according to a regimen that delivers a dose every six weeks. In some embodiments, the dose is administered according to a regimen that delivers a dose every eight weeks.

In some embodiments, the immune checkpoint inhibitor (e.g., an anti-PD-1 antibody such as pembrolizumab or a biosimilar thereof) is administered according to a regimen that delivers a first dose of the immune checkpoint inhibitor for the first 2 to 6 dosing cycles (e.g., the first 3, 4, or 5 dosing cycles), and then delivers a second dose of the immune checkpoint inhibitor for the subsequent dosing cycles until therapy is discontinued (e.g., due to disease progression or an adverse effect or as directed by a physician). In some embodiments, the duration of the first set of 2 to 6 dosing cycles (e.g., the first 3, 4, or 5 dosing cycles) is different from the duration of the subsequent dosing cycles. In some embodiments the first dose and the second dose are the same. In some embodiments, the first dose and the second dose are different.

In an embodiment, the PD-1 antagonist is administered at a dose of 400 mg. In an embodiment, the PD-1 antagonist is administered through infusion Q6W. In an embodiment, the PD-1 antagonist is administered through a 30 minute infusion Q6W

In an embodiment, the PD-1 antagonist is administered at a dose of 200 mg. In an embodiment, the PD-1 antagonist is administered through infusion Q3W. In an embodiment, the PD-1 antagonist is administered through a 30 minute infusion Q3W.

In an embodiment, the pemetrexed is administered at a dose of 500 milligram per meter square (mg/m²). In an embodiment, the pemetrexed is administered Q3W. In an embodiment, the pemetrexed is administered through a 10 minute IV infusion Q3W.

In an embodiment, the cisplatin is administered at a dose of 75 mg/m². In an embodiment, the cisplatin is administered Q3W. In an embodiment, the cisplatin is administered through a 30 minute IV infusion Q3W for 4 cycles.

In an embodiment, the carboplatin is administered at a dose at an area under the concentration time curve of 5 milligram/milliliters/minute (mg/mL/min). In an embodiment, the carboplatin is administered Q3W. In an embodiment, the carboplatin is administered through a 15 to 60 minute IV infusion Q3W for 4 cycles.

In some embodiments, a patient is selected for treatment with an anti-PD-1 antagonist wherein the patient has metastatic NSCLC whose tumors express PD-L1 (TPS≥1%). In an embodiment, the patient has a PD-L1 TPS of 1% or greater. In an embodiment, the patient has a PD-L1 TPS of 10% or greater. In an embodiment, the patient has a PD-L1 TPS of 25% or greater. In an embodiment, the patient has a PD-L1 TPS of 50% or greater. In an embodiment, the patient has a PD-L1 immune cell (IC) score of 10% or greater. In an embodiment, the patient has T cells that express CD137. In an embodiment, the patient has CD8+ T cells that express CD137. In an embodiment, the patient has CD8+ effector T cells (Teff) that express CD137. In an embodiment, the patient has CD8+ memory T cells (Tem) that express CD137. In an embodiment, the patient has CD4+ effector T cells (Teff) that express CD137.

### Dostarlimab and Optional Chemotherapeutic Agents

In some embodiments, the dose of dostarlimab or a biosimilar thereof is within a range of about 5 to about 5000 mg (e.g., about 5 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, about 5000 mg, or a range defined by any two of the foregoing values). In some embodiments, the dose dostarlimab or a biosimilar thereof is 500 mg or 1000 mg.

In some embodiments, the dose is in an amount relative to body weight. In some embodiments, the dose of dostarlimab or a biosimilar thereof is within a range of about 0.01 mg/kg to 100 mg/kg of animal or human body weight; however, doses below or above this exemplary range are within the scope of the disclosure. The daily dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In some embodiments, the dose is administered according to a regimen that delivers a dose every two weeks. In some embodiments, the dose is administered according to a regimen that delivers a dose every three weeks. In some embodiments, the dose is administered according to a regimen that delivers a dose every four weeks. In some embodiments, the dose is administered according to a regimen that delivers a dose every six weeks. In some embodiments, the dose is administered according to a regimen that delivers a dose every eight weeks.

In some embodiments, a composition that delivers dostarlimab or a biosimilar thereof is administered to a patient at a dose of about 1, 3 or 10 mg/kg. In some embodiments, dostarlimab or a biosimilar thereof is administered according to a regimen that delivers a dose of about 1, 3 or 10 mg/kg every two weeks. In some embodiments, dostarlimab or a biosimilar thereof is administered according to a regimen that delivers a dose of about 1, 3 or 10 mg/kg every three weeks. In some embodiments, dostarlimab or a biosimilar thereof is administered according to a regimen that delivers a dose of about 1, 3 or 10 mg/kg every four weeks. In some embodiments, dostarlimab or a biosimilar thereof is administered according to a regimen that delivers a dose of about 1 mg/kg every three weeks. In some embodiments, dostarlimab or a biosimilar thereof is administered according to a regimen that delivers a dose of about 3 mg/kg every three weeks. In some embodiments, dostarlimab or a biosimilar thereof is administered according to a regimen that delivers a dose of about 10 mg/kg every three weeks.

In some embodiments, a composition that delivers dostarlimab or a biosimilar thereof is administered to a patient at a dose of about 400 mg. In some embodiments, dostarlimab or a biosimilar thereof is administered according to a regimen that delivers a dose of about 400 mg every two weeks. In some embodiments, dostarlimab or a biosimilar thereof is administered according to a regimen that delivers a dose of about 400 mg every three weeks. In some embodiments, dostarlimab or a biosimilar thereof is administered according to a regimen that delivers a dose of about 400 mg every four weeks.

In some embodiments, a composition that delivers dostarlimab or a biosimilar thereof is administered to a patient at a dose of about 500 mg. In some embodiments, dostarlimab or a biosimilar thereof is administered according to a regimen that delivers a dose of about 500 mg every two weeks. In some embodiments, dostarlimab or a biosimilar thereof is administered according to a regimen that delivers a dose of about 500 mg every three weeks. In some embodiments, dostarlimab or a biosimilar thereof is administered according to a regimen that delivers a dose of about 500 mg every four weeks.

In some embodiments, a composition that delivers dostarlimab or a biosimilar thereof is administered to a patient at a dose of about 800 mg. In some embodiments, dostarlimab or a biosimilar thereof is administered according to a regimen that delivers a dose of about 800 mg every three weeks. In some embodiments, dostarlimab or a biosimilar thereof is administered according to a regimen that delivers a dose of about 800 mg every four weeks. In some embodiments, dostarlimab or a biosimilar thereof is administered according to a regimen that delivers a dose of about 800 mg every six weeks. In some embodiments, dostarlimab or a biosimilar thereof is administered according to a regimen that delivers a dose of about 800 mg every eight weeks.

In some embodiments, a composition that delivers dostarlimab or a biosimilar thereof is administered to a patient at a dose of about 1,000 mg. In some embodiments, dostarlimab or a biosimilar thereof is administered according to a regimen that delivers a dose of about 1,000 mg every three weeks. In some embodiments, dostarlimab or a biosimilar thereof is administered according to a regimen that delivers a dose of about 1,000 mg every four weeks. In some embodiments, dostarlimab or a biosimilar thereof is administered according to a regimen that delivers a dose of about 1,000 mg every five weeks. In some embodiments, dostarlimab or a biosimilar thereof is administered according to a regimen that delivers a dose of about 1,000 mg every six weeks.

In some embodiments, dostarlimab or a biosimilar thereof is administered according to a regimen that delivers a dose of about 1,000 mg every seven weeks. In some embodiments, dostarlimab or a biosimilar thereof is administered according to a regimen that delivers a dose of about 1,000 mg every eight weeks. In some embodiments, dostarlimab or a biosimilar thereof is administered according to a regimen that delivers a dose of about 1,000 mg every nine weeks.

In some embodiments, dostarlimab or a biosimilar thereof is administered according to a regimen that delivers a dose of about 500 mg every three weeks. In some embodiments, dostarlimab or a biosimilar thereof is administered according to a regimen that delivers a dose of about 1000 mg every six weeks.

In some embodiments, dostarlimab or a biosimilar thereof is administered according to a regimen that delivers a first dose of dostarlimab or a biosimilar thereof for the first 2 to 6 dosing cycles (e.g., the first 3, 4, or 5 dosing cycles), and then delivers a second dose of dostarlimab or a biosimilar thereof for the subsequent dosing cycles until therapy is discontinued (e.g., due to disease progression or an adverse effect or as directed by a physician). In some embodiments, the duration of the first set of 2 to 6 dosing cycles (e.g., the first 3, 4, or 5 dosing cycles) is different from the duration of the subsequent dosing cycles. In embodiments, dostarlimab or a biosimilar thereof is administered according to a regimen that delivers a first dose of dostarlimab or a biosimilar thereof once every three weeks for the first three dosing cycles, and then delivers a second dose of dostarlimab or a biosimilar thereof once every six weeks or more for the remaining dosing cycles (e.g., a second dose of dostarlimab or a biosimilar thereof once every six weeks for the remaining dosing cycles). In embodiments, dostarlimab or a biosimilar thereof is administered according to a regimen that delivers dostarlimab or a biosimilar thereof once every three weeks for the first four dosing cycles, and then delivers a second dose of dostarlimab or a biosimilar thereof once every six weeks or more for the remaining dosing cycles (e.g., a second dose of dostarlimab or a biosimilar thereof once every six weeks for the remaining dosing cycles). In embodiments, dostarlimab or a biosimilar thereof is administered according to a regimen that delivers a first dose of dostarlimab or a biosimilar thereof once every three weeks for the first five dosing cycles, and then delivers a second dose of a dostarlimab or a biosimilar thereof once every six weeks or for the remaining dosing cycles (e.g., a second dose of a dostarlimab or a biosimilar thereof once every six weeks for the remaining dosing cycles). In some embodiments, a dostarlimab or a biosimilar thereof is administered according to a regimen that delivers a first dose of dostarlimab or a biosimilar thereof once every three weeks for the first 2 to 6 dosing cycles (e.g., the first 3, 4, or 5 dosing cycles), and then delivers a second dose of dostarlimab or a biosimilar thereof once every six weeks or until therapy is discontinued (e.g., due to disease progression or an adverse effect or as directed by a physician). In some embodiments, a dostarlimab or a biosimilar thereof is administered according to a regimen that delivers a first dose of dostarlimab or a biosimilar thereof once every three weeks for the first 3, 4, or 5 dosing cycles (e.g., the first 4 dosing cycles), and then delivers a second dose of dostarlimab or a biosimilar thereof once every six weeks or more until therapy is discontinued (e.g., due to disease progression or an adverse effect or as directed by a physician). In embodiments, the method comprises delivering a second dose of dostarlimab or a biosimilar thereof once every six weeks until therapy is discontinued.

In some embodiments the first and/or second dose of dostarlimab or a biosimilar thereof is about 100 mg to about 2,000 mg (e.g., about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg). In some embodiments the first dose and the second dose are the same. In some embodiments, the first dose and the second dose are different. In embodiments, the first dose is about 500 mg of dostarlimab or a biosimilar thereof. In embodiments, the second dose is about 1000 mg of dostarlimab or a biosimilar thereof.

In some embodiments, dostarlimab or a biosimilar thereof is administered according to a regimen that comprises administering about 500 mg dose every 3 weeks for four doses followed by administering at least one about 1,000 mg dose every six weeks after the fourth dose of about 500 mg. In some embodiments, additional about 1,000 mg doses are administered every six weeks after the first about 1000 mg dose until no further clinical benefit is achieved. In some particular embodiments, dostarlimab or a biosimilar thereof is administered according to a dosing regimen that includes 500 mg for 4 cycles Q3W followed by 1000 mg Q6W.

In some embodiments, dostarlimab or a biosimilar thereof is administered according to a regimen that comprises administering a 400 mg dose every 3 weeks for four doses followed by administering at least one 800 mg dose every six weeks after the fourth 400 mg dose. In some embodiments, additional 800 mg doses are administered every six weeks after the first 800 mg dose until no further clinical benefit is achieved. In some particular embodiments, dostarlimab or a biosimilar thereof is administered according to a dosing regimen that includes 400 mg for 4 cycles Q3W followed by 800 mg Q6W.

In an embodiment, the dostarlimab or a biosimilar thereof is administered at a dose of 500 mg. In an embodiment, the dostarlimab or a biosimilar thereof is administered Q3W. In an embodiment, the dostarlimab or a biosimilar thereof is administered through a 30 minute infusion intravenously Q3W. In an embodiment, the dostarlimab or a biosimilar thereof is administered at a dose of 1.000 mg beginning 3 weeks after dose 4. In an embodiment, the dostarlimab or a biosimilar thereof is administered every 6 weeks.

In an embodiment, the patient discontinued administration of at least one of the one or more chemotherapeutic agents prior to beginning administration of dostarlimab or a biosimilar thereof.

In an embodiment, at least one of the one or more chemotherapeutic agents are co-administered with dostarlimab or a biosimilar thereof. In an embodiment, the patient is administered pemetrexed in combination with dostarlimab or a biosimilar thereof. In an embodiment, the pemetrexed is administered at a dose of 500 milligram per meter square (mg/m²). In an embodiment, the pemetrexed is administered Q3W. In an embodiment, the pemetrexed is administered through a 10 minute IV infusion Q3W. In an embodiment, the patient is administered cisplatin in combination with dostarlimab or a biosimilar thereof. In an embodiment, the cisplatin is administered at a dose of 75 mg/m². In an embodiment, the cisplatin is administered Q3W. In an embodiment, the cisplatin is administered through a 30 minute IV infusion Q3W for 4 cycles. In an embodiment, the patient is administered carboplatin in combination with dostarlimab or a biosimilar thereof. In an embodiment, the carboplatin is administered at a dose at an area under the concentration time curve of 5 milligram/milliliters/minute (mg/mL/min). In an embodiment, the carboplatin is administered Q3W. In an embodiment, the carboplatin is administered through a 15 to 60 minute IV infusion Q3W for 4 cycles.

### Measuring Tumor Response

Tumor response can be measured by, for example, the Response Evaluation Criteria in Solid Tumors (RECIST) v1.1 guidelines. The guidelines are provided by E. A. Eisenhauer, et al, “New response evaluation criteria in solid tumors: Revised RECIST guideline (version 1.1.),” Eur. J. of Cancer, 45: 228-247 (2009). In some embodiments. RECIST guidelines may serve as a basis for all protocol guidelines related to disease status. In some embodiments, RECIST guidelines are used to assess tumor response to treatment and/or date of disease progression.

RECIST guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion.

In an embodiment, objective (or overall) response rate (ORR) is evaluated by RECIST v1.1 based on blinded independent central review (BICR) and is defined as the proportion of participants with a best overall response (BOR) of complete response (CR) or partial response (PR) in the analysis population.

In an embodiment, administration of dostarlimab, or a biosimilar thereof, results in improved objective response rate (ORR) relative to administration of the immune checkpoint inhibitor that is not dostarlimab, or a biosimilar thereof, and the one or more chemotherapeutic agents. In an embodiment, administration of dostarlimab, or a biosimilar thereof, results in improved objective response rate (ORR) relative to administration of pembrolizumab or a biosimilar thereof, and the one or more chemotherapeutic agents.

In an embodiment, the method demonstrates non-inferiority to administration of the immune checkpoint inhibitor that is not dostarlimab, or a biosimilar thereof, and the one or more chemotherapeutic agents. In an embodiment, the method demonstrates non-inferiority to administration of pembrolizumab or a biosimilar thereof, and the one or more chemotherapeutic agents. In an embodiment, non-inferiority is demonstrated using objective response rate (ORR). In an embodiment, the ORR is a proportion of participants with a best overall response of confirmed complete response or partial response in an analysis population. In an embodiment, non-inferiority is demonstrated using a process comprising monitoring release of one or more cytokines from one or more tumor explants derived from a patient, i.e., patient-derived tumor explant (PDE). In an embodiment, the PDE is obtained from a patient suffering from or diagnosed with NSCLC. In an embodiment, the patient has been previously treated with a combination of (i) an immune checkpoint inhibitor that is not dostarlimab and (ii) one or more chemotherapeutic agents. In an embodiment, the one or more cytokines include, but are not limited to, IL-2, IFN-γ, interferon gamma-induced protein 10 (IP-10), and interferon-inducible T-cell alpha chemoattractant (I-TAC). In an embodiment, non-inferiority is demonstrated using a process comprising monitoring release of one or more cytokines from PDE including, but not limited to, TNF-α, MIP3-α, IP-10, IL-8, IL-6, IL-2, IL-1β, IL-17A, IL-10, IFN-γ, GRO-alpha, Granzyme B, ENA-78, MIG/CXCL9, BCA-1/CXCL13, and I-TAC/CXCL11.

In an embodiment, the disclosure provides the use of dostarlimab or a biosimilar thereof for the treatment of non-small cell lung cancer (NSCLC) in a patient previously treated with a combination of (i) an immune checkpoint inhibitor that is not dostarlimab or a biosimilar thereof and (ii) one or more chemotherapeutic agents. In an embodiment, the disclosure provides the use of dostarlimab or a biosimilar thereof for the treatment of non-small cell lung cancer (NSCLC) in a patient previously treated with a combination of (i) pembrolizumab or a biosimilar thereof and (ii) one or more chemotherapeutic agents.

In an embodiment, use of dostarlimab, or a biosimilar thereof, results in improved objective response rate (ORR) relative to use of the immune checkpoint inhibitor that is not dostarlimab, or a biosimilar thereof, and the one or more chemotherapeutic agents. In an embodiment, use of dostarlimab, or a biosimilar thereof, results in improved objective response rate (ORR) relative to use of pembrolizumab or a biosimilar thereof, and the one or more chemotherapeutic agents.

In an embodiment, the use demonstrates non-inferiority to use of the immune checkpoint inhibitor that is not dostarlimab, or a biosimilar thereof, and the one or more chemotherapeutic agents. In an embodiment, the use demonstrates non-inferiority to use of pembrolizumab or a biosimilar thereof, and the one or more chemotherapeutic agents. In an embodiment, non-inferiority is demonstrated using objective response rate (ORR). In an embodiment, the ORR is a proportion of participants with a best overall response of confirmed complete response or partial response in an analysis population. In an embodiment, non-inferiority is demonstrated using a process comprising monitoring release of one or more cytokines from one or more tumor explants derived from a patient, i.e., patient-derived tumor explant (PDE). In an embodiment, the PDE is obtained from a patient suffering from or diagnosed with NSCLC. In an embodiment, the patient has been previously treated with a combination of (i) an immune checkpoint inhibitor that is not dostarlimab and (ii) one or more chemotherapeutic agents. In an embodiment, the one or more cytokines include, but are not limited to, IL-2, IFN-γ, IP-10, and I-TAC. In an embodiment, non-inferiority is demonstrated using a process comprising monitoring release of one or more cytokines from PDE including, but not limited to, TNF-α, MIP3-α, IP-10, IL-8, IL-6, IL-2, IL-10, IL-17A, IL-10, IFN-γ, GRO-alpha, Granzyme B, ENA-78, MIG/CXCL9, BCA-1/CXCL13, and I-TAC/CXCL11.

The following examples are provided to illustrate, but not limit the claimed disclosure.

### EXAMPLES

### Example 1: Randomized, Phase 2, Double-Blind Study to Evaluate the Efficacy of Dostarlimab Plus Chemotherapy Versus Pembrolizumab Plus Chemotherapy in Metastatic Non-Squamous Non-Small Cell Lung Cancer (NSCLC)

A study comparing the efficacy and safety PD-1 inhibitors dostarlimab and pembrolizumab, when administered in combination with chemotherapy (pemetrexed, cisplatin and carboplatin), in participants with non-squamous NSCLC without a known sensitizing epidermal growth factor receptor (EGFR), anaplastic lymphoma kinase (ALK), or receptor tyrosine kinase-1 (ROS-1) mutation, BRAF V600E mutation, or other genomic aberration for which an approved targeted therapy is available was conducted. A total of approximately 240 participants were enrolled in the study estimated to be completed in a period of 5 years.

### Arms and Interventions

### Experimental Arm: Participants Receiving Dostarlimab Plus Chemotherapy.

Participants will receive dostarlimab on Day 1 of every 21 Day cycle followed by pemetrexed, and then followed by cisplatin or carboplatin (Cycles 1 to 4 only) as per investigator decision.

Drug: Dostarlimab will be administered through a 30 minute infusion at a dose of 500 milligrams (mg) intravenously (IV) every 3 weeks (Q3W) up to a maximum of 35 cycles (each cycle of 21 days).

Chemotherapy: Pemetrexed will be administered at 500 milligram per meter square (mg/m²) IV through a 10 minute IV infusion Q3W, up to a maximum of 35 cycles (each cycle of 21 days). Cisplatin will be administered at 75 mg/m² through a 30 minute IV infusion Q3W for 4 cycles (each cycle of 21 days) as per investigator decision. Carboplatin will also be administered at area under the concentration time curve 5 milligram/milliliters/minute (mg/mL/min) (maximum dose: 750 mg) through a 15 to 60 minute IV infusion Q3W for 4 cycles (each cycle of 21 days) as per investigator decision.

### Active Comparator Arm: Participants Receiving Pembrolizumab Plus Chemotherapy.

Participants will receive pembrolizumab on Day 1 of every 21 Day cycle followed by pemetrexed, and then followed by cisplatin or carboplatin (Cycles 1 to 4 only) as per investigator decision.

Drug: Pembrolizumab will be administered through a 30 minute infusion at a dose of 200 mg Q3W up to a maximum of 35 cycles (each cycle of 21 days).

Chemotherapy: Pemetrexed will be administered at 500 milligram per meter square (mg/m²) IV through a 10 minute IV infusion Q3W, up to a maximum of 35 cycles (each cycle of 21 days). Cisplatin will be administered at 75 mg/m² through a 30 minute IV infusion Q3W for 4 cycles (each cycle of 21 days) as per investigator decision. Carboplatin will also be administered at area under the concentration time curve 5 milligram/milliliters/minute (mg/mL/min) (maximum dose: 750 mg) through a 15 to 60 minute IV infusion Q3W for 4 cycles (each cycle of 21 days) as per investigator decision.

### Outcome Measures

### Primary Outcome Measures:

1. Overall Response Rate (ORR) by Response Evaluation Criteria in Solid Tumors (RECIST) version 1.1 (v1.1). [Time Frame: Up to 5 years]. ORR will be evaluated by RECIST v1.1 based on blinded independent central review (BICR) and will be defined as the proportion of participants with a best overall response (BOR) of complete response (CR) or partial response (PR) in the analysis population.

### Secondar Outcome Measures:

1. Overall survival (OS) [Time Frame: Up to 5 years]. OS is defined as the time from the date of randomization to the date of death by any cause.
2. Progression free survival (PFS) by RECIST v1.1 [Time Frame: Up to 5 years]. PFS will be evaluated using RECIST v1.1 based on Investigator assessment and is defined as the time from the date of randomization to the date of progressive disease (PD) or death by any cause, whichever occurs first.
3. Number of participants with treatment-emergent adverse events (TEAEs) [Time Frame: Up to 5 years]. A TEAE is any event that was not present prior to the initiation of study treatment or any event already present that worsens in either intensity or frequency following exposure to study treatment.
4. Number of participants with serious adverse events (SAEs) [Time Frame: Up to 5 years]. An SAE is any untoward medical occurrence that, at any dose results in death, is life-threatening, requires inpatient hospitalization or prolongation of existing hospitalization, results in persistent or significant disability or incapacity, is a congenital anomaly/birth defect or is an important medical event that may jeopardize the participant or may require medical or surgical intervention to prevent one of the above outcomes.
5. Number of participants with immune related adverse events (irAEs) [Time Frame: Up to 5 years]. The irAEs are events which may be severe or fatal and can occur in participants treated with monoclonal antibodies directed against immune checkpoints, including pembrolizumab and dostarlimab. While irAEs (e.g., diarrhea/colitis, pneumonitis, nephritis, hypophysitis, adrenalitis, thyroiditis, severe skin reactions, uveitis, myocarditis, and hepatotoxicity) usually occur during treatment, symptoms can also manifest after discontinuation of treatment.
6. Number of participants with TEAEs leading to death [Time Frame: Up to 5 years]. Number of participants with TEAEs leading to death will be assessed.
7. Number of participants with adverse events leading to discontinuation (AELD) [Time Frame: Up to 5 years]. Number of participants with AELDs will be assessed.
8. Number of participants with clinically significant changes in hematology, clinical chemistry, thyroid function and urinalysis lab parameters. [Time Frame: Up to 5 years]. Blood and urine samples will be collected to evaluate hematology, clinical chemistry, thyroid function and urinalysis lab parameters.
9. Number of participants with abnormal vital signs [Time Frame: Up to 5 years]. Number of participants with abnormal vital signs will be assessed.
10. Number of participants with abnormal Eastern Cooperative Oncology Group (ECOG) performance status [Time Frame: Up to 5 years]. Performance status will be assessed using the ECOG scale (Grade 0-4). Grade 0 indicates fully active, able to carry on all pre-disease performance without restriction and Grade 4 indicates completely disabled, cannot carry on any self-care and totally confined to bed or chair.
11. Number of participants with abnormal electrocardiogram (ECG) parameters [Time Frame: Up to 5 years]. Participants will be supine or in a semi-recumbent position and rested for approximately 2 minutes before ECGs are recorded.
12. Number of participants with abnormal physical examination [Time Frame: Up to 5 years]. Physical examination will include assessments of the cardiovascular, respiratory, gastrointestinal, and neurological systems. Height and weight will also be measured and recorded.
13. Number of participants receiving concomitant medications [Time Frame: Up to 5 years]. Concomitant medications will be recorded.

### Eligibility Criteria

### Inclusion Criteria:

Participant must be greater than equal to (>=) 18 years old, must be able to understand the study procedures, and agrees to participate in the study by providing written informed consent which includes compliance with the requirements and restrictions listed in the informed consent form (ICF) and in this protocol.

Participant has histologically- or cytologically-confirmed metastatic non-squamous NSCLC with documented absence of a sensitizing EGFR, ALK, ROS-1, or BRAFV600E mutation or other genomic aberration for which an approved targeted therapy is available. Mixed tumors will be categorized by the predominant cell type; if the tumor has predominantly squamous cell histology or if small cell elements are present, the participant is ineligible.

Participants must have measurable disease, that is (i.e.) presenting with at least 1 measurable lesion per RECIST v1.1 as determined by the local site Investigator/radiology assessment. Target lesions situated in a previously irradiated area are considered measurable if progression has been demonstrated in such lesions and if there are other target lesions. If there is only 1 target lesion that was previously irradiated, the participant is not eligible.

Participant has documented PD L1 status by the 22C3 pharmDx assay (Agilent/Dako). If no prior PD L1 result is available at the time of Screening, the participant can be tested locally using the stated method, or central PD L1 testing can be completed. Results are needed for stratification and must be available prior to randomization.

Participant has an ECOG performance status score of 0 or 1.

Participant has a life expectancy of at least 3 months.

Participant has adequate organ function.

Participant has recovered to Grade less than equal to (<=)1 from any prior treatment related toxicities at the time of randomization. A participant with Grade 2 alopecia is an exception to this criterion and may qualify for this study.

Contraceptive use by male and female participants should be consistent with local regulations regarding the methods of contraception for those participating in clinical studies.

Male participants are eligible to participate if they agree to the following during the Treatment Period and for at least 150 days after the last dose of study treatment:

Refrain from donating sperm plus, either:

Be abstinent from heterosexual intercourse as their preferred and usual lifestyle (abstinent on a long term and persistent basis) and agree to remain abstinent.

Must agree to use contraception/barrier as follows:

Agree to use a male condom (and should also be advised of the benefit for a female partner to use a highly effective method of contraception, as a condom may break or leak) when having sexual intercourse with a woman of childbearing potential (WOCBP) who is not currently pregnant.

Agree to use a male condom when engaging in any activity that allows for passage of ejaculate to another person.

A female participant is eligible to participate if she is not pregnant or breastfeeding, and 1 of the following conditions applies:

Is a woman of non childbearing potential (WONCBP).

Is a WOCBP, using a contraceptive method that is highly effective (with a failure rate of <1% per year and, preferably, with low user dependency) during the Treatment Period and for at least 180 days after the last dose of study treatment and agrees not to donate eggs (ova or oocytes) for the purpose of reproduction during this period. The Investigator should evaluate the potential for contraceptive method failure (for example [e.g.], noncompliance and recently initiated) in relationship to the first dose of study treatment.

A WOCBP must have a negative highly sensitive pregnancy test (urine or serum, as required by local guidelines) within 72 hours before the first dose of study treatment. If a urine test cannot be confirmed as negative (e.g., an ambiguous result), a serum pregnancy test is required. In such cases, the participant must be excluded from participation if the serum pregnancy result is positive.

### Exclusion Criteria:

Participant has received prior systemic therapy for the treatment of metastatic NSCLC. Participants who have received neoadjuvant or adjuvant chemotherapy are eligible if the neoadjuvant/adjuvant therapy was completed at least 12 months prior to the development of metastatic disease.

Participant has received prior therapy with a PD (L)1 or PD L2 inhibitor, a cytotoxic T lymphocyte associated protein 4 (CTLA 4) inhibitor, a T cell immunoglobulin and mucin domain containing 3 (TIM 3) inhibitor, or any other immunotherapy agent (e.g., OX40) for the treatment of cancer.

Participant has received radiation to the lung that is >30 Gray (Gy) within 6 months of the first dose of study treatment.

Participant has completed palliative radiotherapy within 7 days of the first dose of study treatment.

Participant is ineligible if any of the following hepatic characteristics are present:

Alanine aminotransferase (ALT)>2.5 times upper limit of normal (ULN) without liver metastases/tumor infiltration.

ALT>5 times ULN with liver metastases/tumor infiltration.

Bilirubin>1.5 times ULN (isolated bilirubin>1.5 times ULN is acceptable if bilirubin is fractionated and direct bilirubin is <35%).

Current active liver or biliary disease (with the exception of Gilbert's syndrome or asymptomatic gallstones, liver metastases, or otherwise stable chronic liver disease per Investigator assessment).

Participant has a corrected QT interval (QTc)>450 milliseconds (msec) (or QTc>480 msec for participants with bundle branch block).

Participant has had major surgery within 3 weeks of the first dose of study treatment or has not adequately recovered from any AEs (Grade<=1) and/or complications from any major surgery. Surgical implantation of a port catheter is not exclusionary.

Participant has an additional malignancy or a history of prior malignancy, with the exception of adequately treated basal or squamous skin cancer, cervical carcinoma in situ, or bladder carcinoma in situ without evidence of disease, or had a malignancy treated with curative intent and with no evidence of disease recurrence for 5 years since the initiation of that therapy.

Participant has known active brain metastases and/or leptomeningeal metastases. Participants who have received prior therapy for their brain metastases and have radiographically stable central nervous system disease may participate, provided they are neurologically stable for at least 2 weeks before study entry and must be off corticosteroids within 3 days prior to the first dose of study treatment. Stable brain metastases by this definition should be established prior to the first dose of study treatment. Participants with known untreated, asymptomatic brain metastases (i.e., no neurological symptoms, no requirements for corticosteroids, no or minimal surrounding edema, and no lesions>1.5 centimeters [cm]) may participate, but will require regular imaging of the brain as a site of disease.

Participant has tested positive for the presence of hepatitis B surface antigen or has a positive hepatitis C antibody test result at Screening, or within 3 months prior to first dose of study treatment. For potent immunosuppressive agents, participants who test positive for the presence of hepatitis B core antibody should also be excluded.

Participant has an active infection requiring systemic therapy within 1 week prior to the anticipated first dose of study treatment.

Participant has known human immunodeficiency virus (HIV) (positive for HIV 1 or HIV 2 antibodies).

Participant has active autoimmune disease that required systemic treatment in the past 2 years, is immunocompromised in the opinion of the Investigator, or is receiving systemic immunosuppressive treatment. Replacement therapy (e.g., thyroxine, insulin, or physiologic corticosteroid replacement therapy for adrenal or pituitary insufficiency) is not considered a form of systemic treatment.

Participant has received systemic steroid therapy within 3 days prior to the first dose of the study treatment or is receiving any other form of immunosuppressive medication. Replacement therapy is not considered a form of systemic therapy. Use of inhaled corticosteroids, local steroid injection, or steroid eye drops is allowed.

Participant has symptomatic ascites or pleural effusion. A participant who is clinically stable following treatment of these conditions (including therapeutic thoraco or paracentesis) is eligible.

Participant has current interstitial lung disease, current pneumonitis, or a history of pneumonitis that required the use of oral or IV glucocorticoids to assist with management. Lymphangitic spread of the NSCLC is not exclusionary.

Participant has a history or current evidence of any medical condition, therapy, or laboratory abnormality that might confound the study results, interfere with their participation for the full duration of the study treatment, or indicate it is not in the best interest of the participant to participate, in the opinion of the Investigator.

Participant has clinically active diverticulitis, intra-abdominal abscess, gastrointestinal obstruction, or peritoneal carcinomatosis.

Participant has preexisting peripheral neuropathy that is Grade>=2 by National Cancer Institute Common Terminology Criteria for Adverse Events (NCI CTCAE) v5.0 criteria.

Participant has received a live vaccine within 30 days of the first dose of study treatment. Seasonal flu vaccines that do not contain live virus are permitted.

Participant does not meet requirements per local prescribing guidelines for receiving treatment with either pemetrexed and cisplatin or carboplatin.

Participant has sensitivity to any of the study treatments, or components thereof, or a history of drug or other allergy that, in the opinion of the Investigator or GlaxoSmithKline (GSK) Medical Monitor, contraindicates their participation.

Participant is unable to interrupt aspirin or other nonsteroidal anti-inflammatory drugs (NSAIDs), other than an aspirin dose<=1.3 gram (g) per day, for a 5 day period (8 day period for long acting agents, such as piroxicam).

### Methods and Results

Patients with no known EGFR, ALK or genomic aberrations actionable locally-by targeted therapies, known PD-L1 status, ECOG performance status 0 or 1 and no prior systemic treatment for metastatic NSCLC were randomized 1:1 to dostarlimab 500 mg or pembrolizumab 200 mg Q3W IV 35 cycles, both in combination with chemotherapy (4 cycles pemetrexed (500 mg/m²) plus carboplatin (AUC 5 mg/mL/min) or cisplatin (75 mg/m²) then pembrolizumab up to cycle 35) Q3W IV. The primary endpoint was ORR by BICR (RECIST V1.1: difference by Mantel and Haenszel test and Sato's variance estimator; point estimates by Clopper-Pearson). Safety was a secondary endpoint. Disease assessments were at weeks 6 and 12, then every 9 weeks×4, then every 12 weeks.

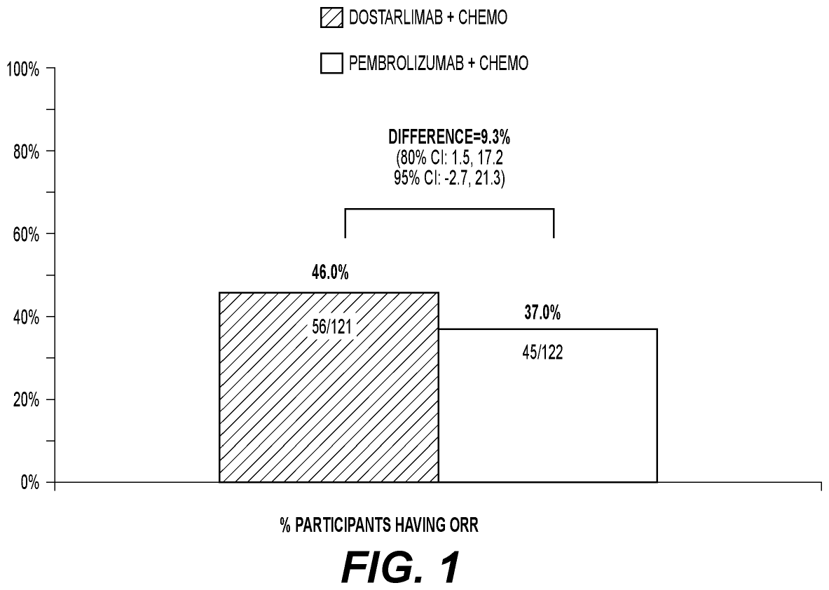

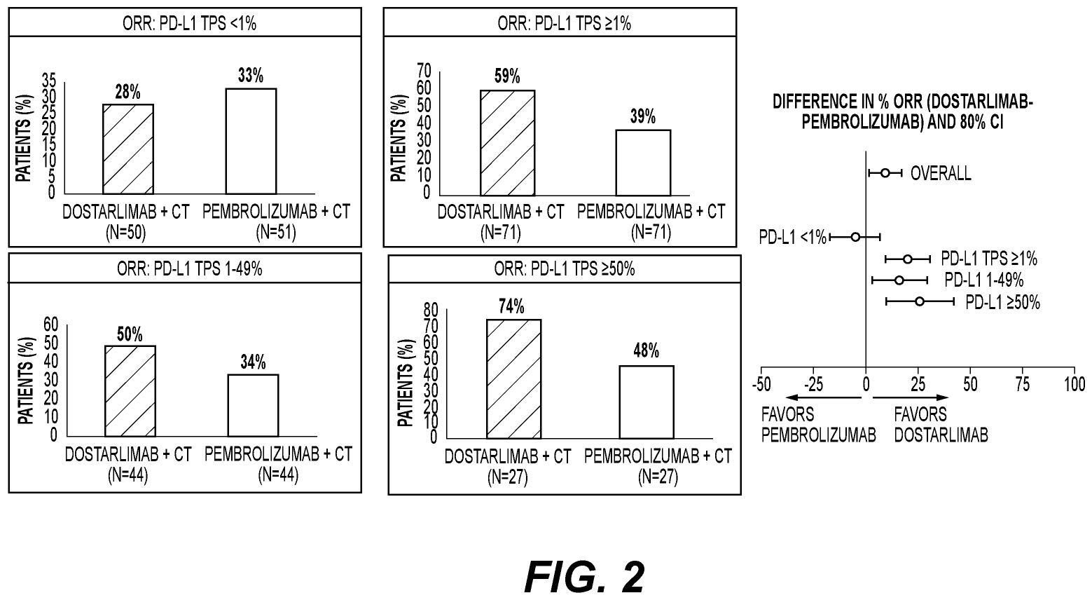

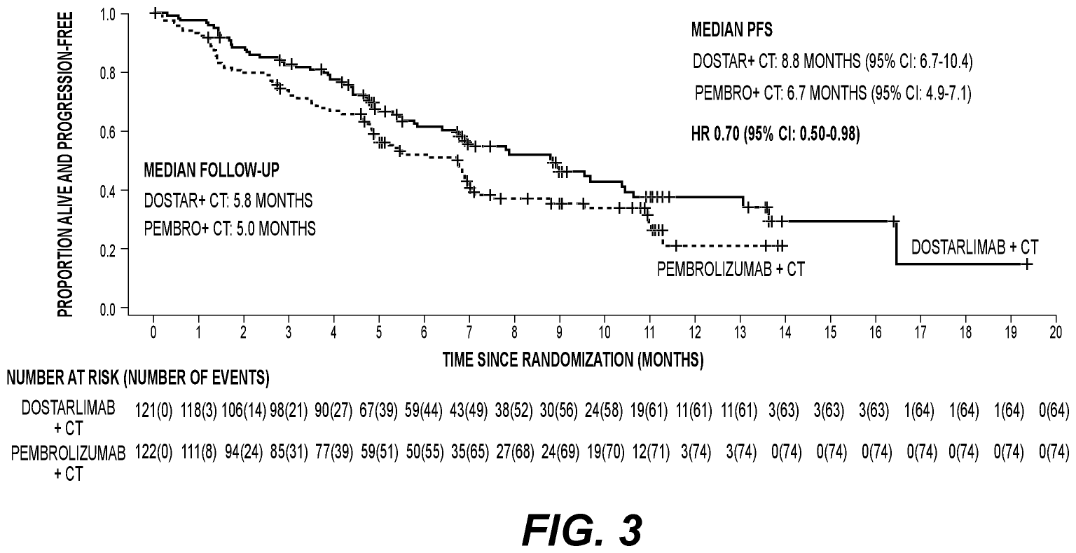

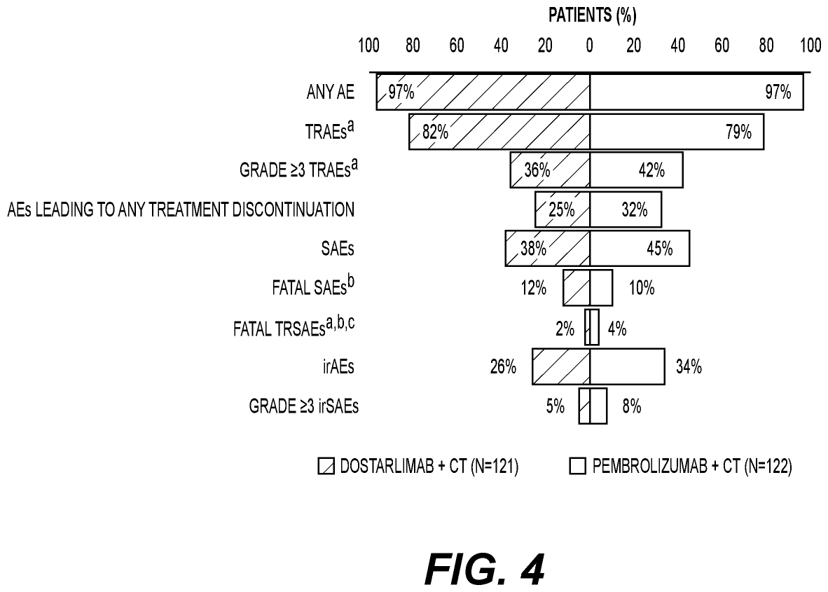

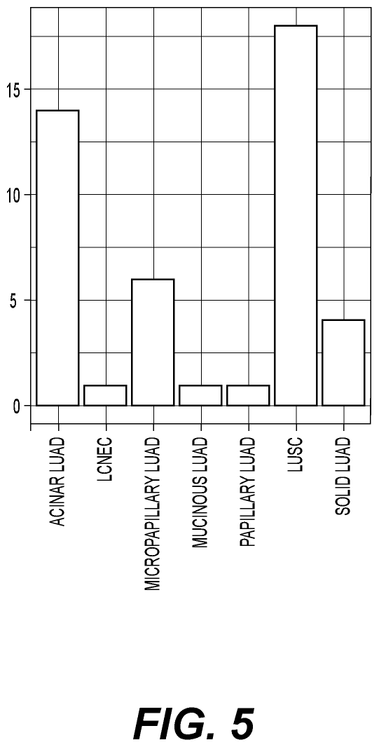

In total, 121 and 122 patients in the dostarlimab plus chemotherapy and pembrolizumab plus chemotherapy arms were treated and evaluable, respectively. Overall, 41% and 42% of patients had PD-L1 tumor proportion score (TPS)<1; 36% had TPS 1-49% and 22% had TPS 50 in both arms. ORR was 46% for dostarlimab plus chemotherapy, with two complete responses (CRs) (2%) and 54 partial responses (PRs) (45%); ORR was 37% for pembrolizumab plus chemotherapy, with three CRs (2%) and 42 PRs (34%) (9.3% difference (95% CI: −2.7-21.3)). The ORR results are shown in FIG. 1. Safety is shown in Table 2 and FIG. 4. The proportion of patients experiencing treatment-related adverse events (TRAEs) was similar for patients treated with dostarlimab plus chemotherapy and patients treated with pembrolizumab plus chemotherapy (FIG. 4). A numerical trend in the number of Grade 23 TRAEs, adverse events (AEs) leading to treatment discontinuation, serious adverse events (SAEs), and immune-related adverse events (irAEs) in favor of dostarlimab was noted between groups (FIG. 4). The most common TRAEs (>15% in either group) were anemia, asthenia, nausea, and neutropenia. PFS (secondary endpoint) (FIG. 3), PD-L1 sub-analyses (FIG. 2), and duration of response (DOR) (exploratory endpoint) are also analyzed. Efficacy was similar in PD-L1 subgroups, with numerically higher ORR for dostarlimab plus chemotherapy than pembrolizumab plus chemotherapy in patients with PD-L1 TPS≥1%. 1-49%, and ≥50% (FIG. 2). Patients treated with dostarlimab plus chemotherapy had a median PFS of 8.8 months (95% CI: 6.7-10.4), and a median follow-up of 5.8 months, while patients treated with pembrolizumab plus chemotherapy had a median PFS of 6.7 months (95% CI: 4.9-7.1), and a median follow-up of 5.0 months (FIG. 3). Patients treated with dostarlimab plus chemotherapy had a mean DOR of 8.8 months (95% CI: 6.3-13.6), while for patients treated with pembrolizumab plus chemotherapy, DOR was not reached (95% CI: 7.7 months-not reached).

TABLE 2

| | Dostarlimab | Pembrolizumab |
|---|---|---|
| | plus | plus |
| | chemotherapy | chemotherapy |
| n, (%) | (n = 121) | (n = 122) |
| TEAEs | 117 (97) | 118 (97) |
| Grade ≥3 | 71 (59) | 73 (60) |
| Serious | 46 (38) | 54 (44) |
| Leading to any treatment- | 30 (25) | 39 (32) |
| discontinuation* | | |
| Leading to dosarlimab/ | 18 (15) | 29 (24) |
| pembrolizumab treatment- | | |
| discontinuation* | | |
| Leading to death | 15 (12) | 12 (10) |

*Permanent treatment-discontinuation.
TEAE, treatment-emergent adverse event

These results demonstrate that in this first randomized Phase II study to directly compare PD-1 inhibitors, dostarlimab plus chemotherapy showed comparable efficacy to pembrolizumab plus chemotherapy in (first-line) 1L metastatic non-squamous non-oncogenic NSCLC. Safety profiles were similar and consistent with published data.

### Example 2: Comparison of Dostarlimab with Pembrolizumab in a Patient-Derived Tumor Explant (PDE) Model of Non-Small Cell Lung Cancer (NSCLC)

### Production of Patient-Derived Tumor Explant (PDE) Model

Live NSCLC tissue from treatment-naïve NSCLC patients were collected after surgery and the preparation for the PDE model was initiated. Tumor pieces were embedded in low-melting agarose and cut into 250-μm thick precision-cut slices (PCS) using a Krumdieck tissue slicer. PCS were randomized and transferred into culture inserts placed in 6-well culture plates containing culture medium. Then, PCS were treated with dostarlimab, pembrolizumab or isotype controls (4 wells per treatment). After 48 hours incubation, supernatant and PCS were collected prior to cytokine level measurement (MesoScale Discovery). Tumor tissues were extensively profiled prior to (baseline) and after (endpoint) PDE culture using multiple analysis modalities to generate a comprehensive tumor and treatment response profile. Standard baseline readouts encompassed quantitative immunohistochemistry combined with exploratory single cell (flow cytometry) and additional multi-omics approaches for deep immune phenotyping. Cytokine level measurement (MesoScale Discovery) was employed as a functional readout of tumor micro environment (TME) immune cell activation.

The PDE model was optimized to preserve cell type composition throughout the in vitro culture time. The main immune cell lineages, including NK-T, CD4+ and CD8+ T cells, macrophages, B and plasma cells, NK cells and dendritic cells, were present and viable in the PCS cultured for up to 3 days. 3D imaging analysis (live confocal imaging) showed that cells were viable across the full thickness of the tissue slices (250 μm). Complex intratumoral structures such as tertiary lymphoid structures (TLS) were present in the PCS and preserved for the duration of the in vitro culturing (as measured by immunohistochemistry (IHC) analysis).

### PDE Dataset

The PDE dataset consists of N=54 NSCLC donors. Donor and tumor characteristics included in the PDE dataset are provided in Table 3. These characteristics include: main diagnosis (LCNEC, Large cell neuroendocrine carcinoma; LUAD, lung adenocarcinoma; LUSC, lung squamous cell carcinoma), tumor stage (I early stage to IV advanced/metastatic), grading of the resected tumor (G2 moderately differentiated: G3 poorly differentiated), smoking status (ex, former smoker; never smoker; current smoker), PD-L1 TPS (tumor proportion score, determined by IHC), and tumor mutational burden (TMB). The distribution of tumor subtypes (absolute count) is shown in FIG. 5.

TABLE 3

| | Proportion in % |
|---|---|
| Characteristics | (No. of donor or tumor) |
| Gender | 33% Female (N = 18) |
| | 67% Male (N = 36) |
| Diagnosis | 54% LUAD (N = 29) |
| | 39% LUSC (N = 21) |
| | 2% LCNEC (N = 1) |
| | (unknown N = 3) |
| Smoking | 61% ex (N = 33) |
| Status | 11% never (N = 6) |
| | 28% active (N = 15) |
| Grading | 48% G2 (N = 26) |
| | 37% G3 (N = 20) |
| | (unknown N = 8) |
| Tumor | 17% I (N = 9) |
| stage | 24% II (N = 13) |
| | 41% III (N = 22) |
| | 4% IV (N = 2) |
| | (unknown N = 8) |
| PD-L1 | 33% < 1% (N = 18) |
| TPS | 41% 1-49% (N = 22) |
| | 19% ≥ 50% (N = 10) |
| | (unknown N = 4) |
| PD-L1 | 76% < 10% (N = 41) |
| IC score | 19% ≥ 10% (N = 10) |
| | (unknown N = 3) |
| TMB | 94% > 10 mut/Mb (N = 51) |
| | 4% < 10 mut/Mb (N = 2) |
| | (unknown N = 1) |

TMB: tumor mutational burden, TPS: tumor proportion score, IC score: immune cell score.

The TMB was calculated based on INVIEW Oncoprofiling data (Eurofins Genomics). The percentage of CD8+ cells was determined using IHC. The PD-L1 TPS and PD-L1 immune cell score (IC) were also derived from IHC. All-comers were included in this analysis and no sub-selection based on the PD-L1 TPS was made. In general, this PDE dataset contains more male (67%) than female (37%) donors. The distribution of the main diagnosis was in line with expected proportions among NSCLC patients, with 54% LUAD, 39% LUSC and 2% LCNEC. Most donors were either active or former smokers (89%). The distribution of tumor stages was 41% stage III, 24% stage II, 17% stage I and 4% stage IV.

### Results of Dostarlimab and Pembrolizumab Treatment

### Immune Activation Following Dostarlimab Treatment:

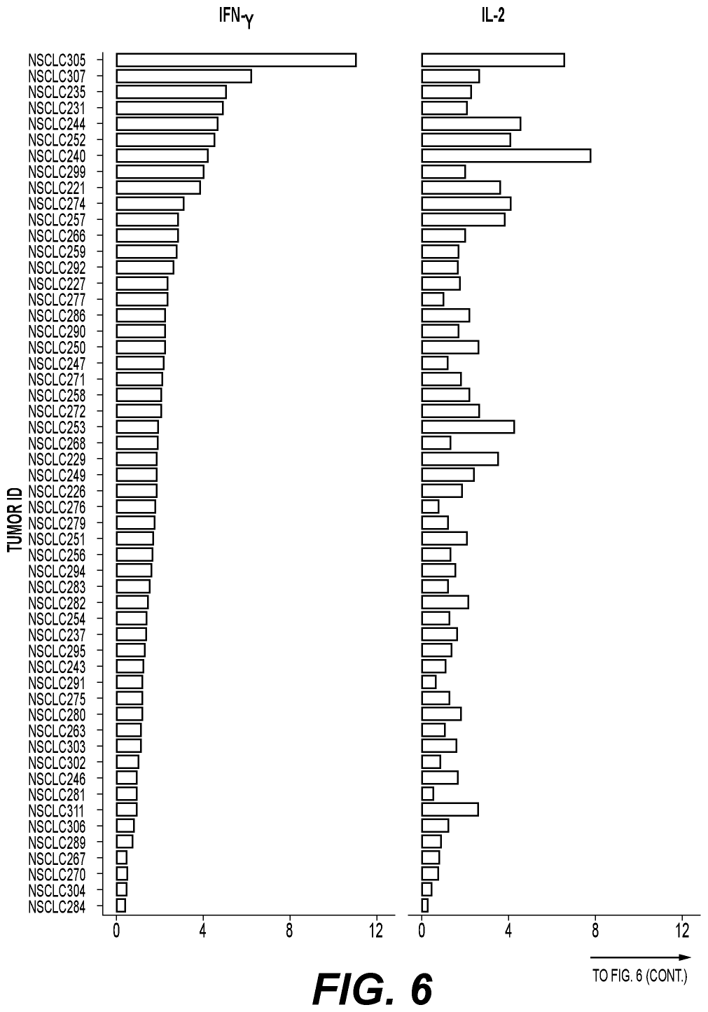

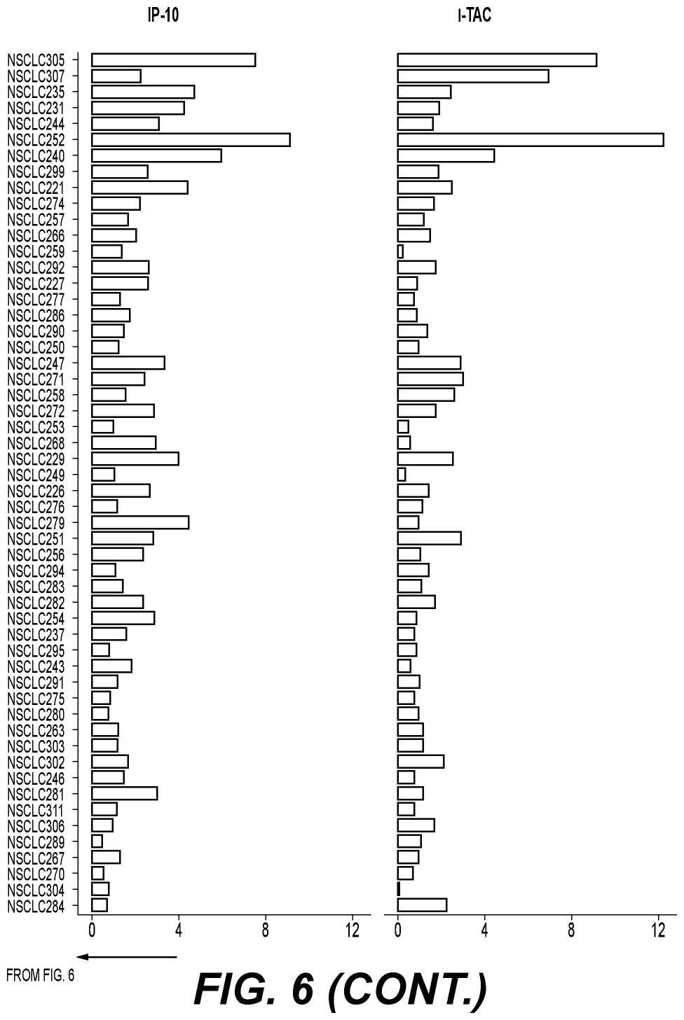

The intratumoral immune activation following treatment with dostarlimab was assessed in N=54 NSCLC donors. Interferon-gamma (IFN-γ) and interleukin-2 (IL-2) release, along with other cytokines and chemokines, were measured as indicators of immune activation. Fold changes of secreted IL-2, IFN-γ, IP-10, and I-TAC relative to human IgG controls are shown in FIG. 6. The results show that NSCLC tumors demonstrate differential immune activation following dostarlimab treatment.

### Comparison of Dostarlimab to Pembrolizumab in Immune Activation:

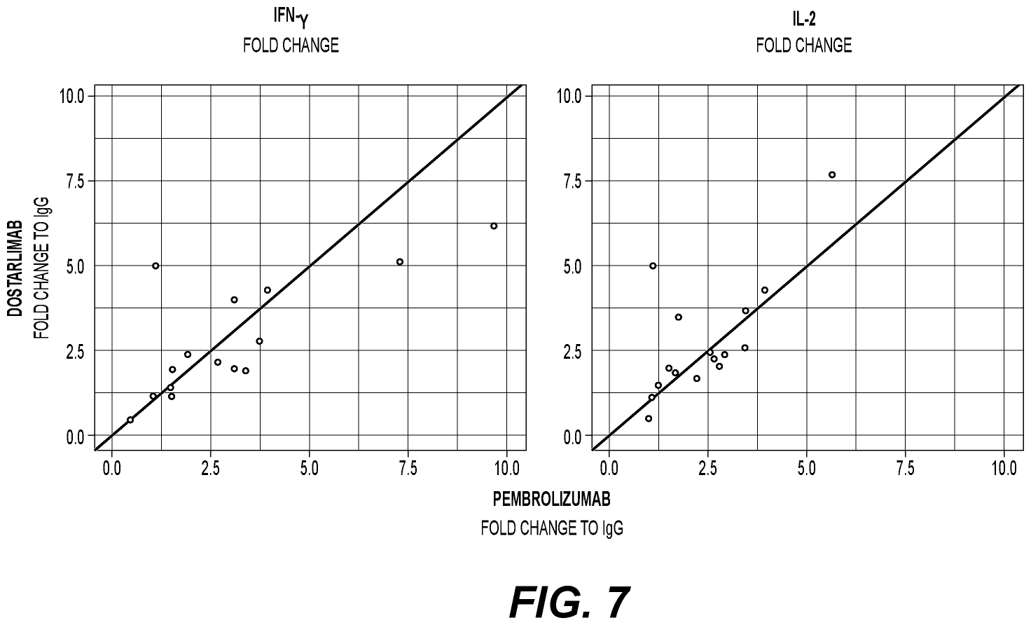

Dostarlimab and pembrolizumab were tested in parallel in explants derived from N=15 NSCLC donors at single concentrations selected based on the estimated concentrations at the tumor site after dosing patients with 500 mg Q3W for dostarlimab and 200 mg Q3W for pembrolizumab, resulting in 18 μg/mL and 10 μg/mL, respectively. Secreted IL-2 and IFN-γ were then quantified as measures of immune activation. The levels of these cytokines were modelled separately using a linear mixed model with treatment as a main effect and accounting for donor-to-donor variability; modelling was done at the technical replicate level with 2-4 replicates per donor. Post-hoc equivalence tests were performed using the resulting marginal means, i.e. two one-sided t-tests were performed. The results indicated that the functional response to dostarlimab in the PDE NSCLC dataset was similar to pembrolizumab for IL-2 (p=0.0189) and IFN-γ (p=0.0521) within the range set up [0.75-1.33] for the dostarlimab vs pembrolizumab ratio (FIG. 7). Thus, the response to dostarlimab is similar to the response to pembrolizumab in the NSCLC PDE model.

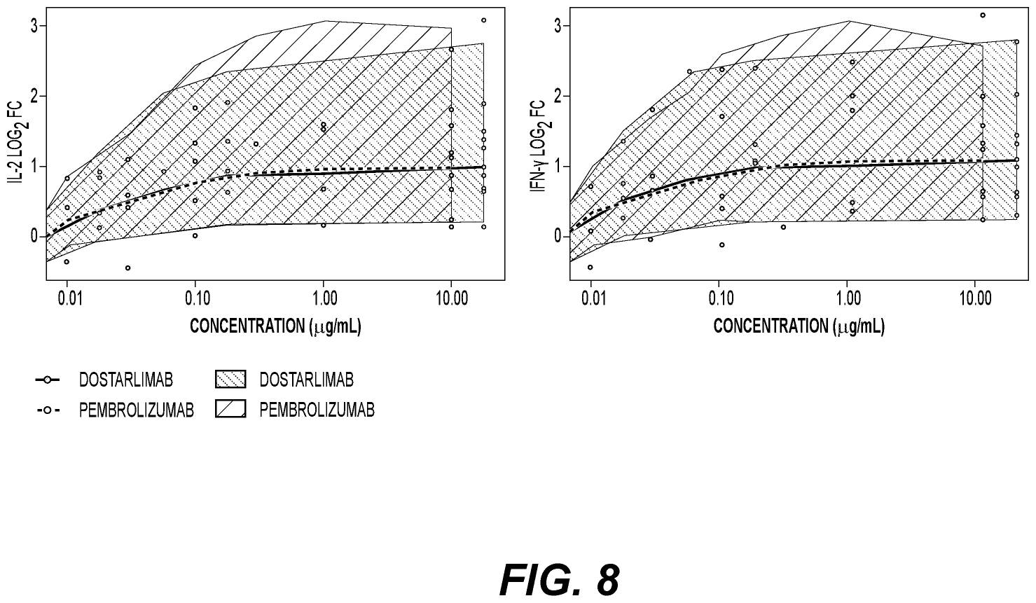

Concentration-dependent induction of immune activation was assessed for pembrolizumab and dostarlimab in N=12 NSCLC PDE using nonlinear mixed effect modeling. The nonlinear mixed effect model described the log 2-fold change (log 2FC) from baseline IgG control of secreted interleukin-2 (IL-2) and interferon-gamma (IFN-γ) relative to PD-1 antagonist concentration (FIG. 8). Treatment with dostarlimab or pembrolizumab was introduced as a covariate on the concentration resulting in the half-maximal induction (EC₅₀). Including the treatment effects resulted in no significant changes to minus twice the log-likelihood (-2LL) describing the IL-2 or the IFN-γ response data (p=0.796 and p=0.430, respectively) such that the data does not support a difference in the IL-2 EC₅₀ or the IFN-γ EC₅₀ between dostarlimab and pembrolizumab (FIG. 8; Tables 4 and 5).

TABLE 4
Estimate for the parameters of the non-linear mixed-
effect sigmoid model, without treatment as covariate.

| | Estimate | | Estimate | |
|---|---|---|---|---|
| | (% CV) | IIV (%) | (% CV) | IIV (%) |
| Parameter | [95% CI] | [% CV] | [95% CI] | [% CV] |
| (units) | for IL-2 | for IL-2 | for IFN-γ | for IFN-γ |
| E₀ | 0 (fixed) | — | 0 (fixed) | — |
| (log₂FC) | | | | |
| E_{MAX} | 0.996 (22) | 66.3 [57] | 1.12 (20) | 63.9 [44] |
| (log₂FC) | [0.571-1.42] | | [0.675-1.56] | |
| EC₅₀ | 0.0305 (20) | — | 0.0238 (22) | — |
| (ug/mL) | └0.0183- | | └0.0137- | |
| | 0.0427┘ | | 0.0339┘ | |
| H (—) | 1 (fixed) | — | 1 (fixed) | — |

| | | |
|---|---|---|
| Add. Error | 0.219 (24) | 0.287 (31) |
| (log₂FC) | | |
| AIC | −71.338 | −43.91 |
| −2LL | −79.338 | −51.91 |

−2LL: minus twice the log-likelihood, Add. Error: additive error, AIC: Akaike Information criterion, CV: coefficient of variation, CI: confidence interval, IIV: inter-explant variability, FC: fold change, E₀: baseline induction, EC₅₀: drug concentration resulting in the half maximal induction, EMAX: maximal cytokine induction, H: Hill coefficient, log₂FC: log₂-fold change.

TABLE 5
Estimate for the parameters of the non-linear mixed-
effect sigmoid model, with treatment as covariate.

| | Estimate | | Estimate | |
|---|---|---|---|---|
| | (% CV) | IIV (%) | (% CV) | IIV (%) |
| Parameter | [95% CI] | [% CV] | [95% CI] | [% CV] |
| (units) | for IL-2 | for IL-2 | for IFN-γ | for IFN-γ |
| E₀ | 0 (fixed) | — | 0 (fixed) | — |
| (log₂FC) | | | | |
| E_{MAX} | 0.997 (22) | 66.1 [57] | 1.13 (20) | 63.3 [44] |
| (log₂FC) | [0.576-1.42] | | [0.687-1.57] | |
| EC₅₀ | 0.0288 (19) | — | 0.0207 (15) | — |
| (ug/mL) | [0.0179- | | [0.0146- | |
| | 0.0397] | | 0.0268] | |
| H (—) | 1 (fixed) | — | 1 (fixed) | — |
| Pembrolizumab | 9.95 (290) | — | 31.1 (69) | — |
| on EC₅₀ (%) | [−47.3-67.2] | | [−10.8-73.0] | |

| | | |
|---|---|---|
| Add. Error | 0.219 (24) | 0.285 (31) |
| (log₂FC) | | |
| AIC | −69.405 | −42.533 |
| −2LL | −79.405 | −52.533 |

−2LL: minus twice the log-likelihood, Add. Error: additive error, AIC: Akaike Information criterion, CV: coefficient of variation, CI: confidence interval, IIV: inter-explant variability, FC: fold change, E₀: baseline induction, EC₅₀: drug concentration resulting in the half maximal induction, EMAX: maximal cytokine induction, H: Hill coefficient, log₂FC: log₂-fold change.

### Response of NSCLC Subgroups to Dostarlimab:

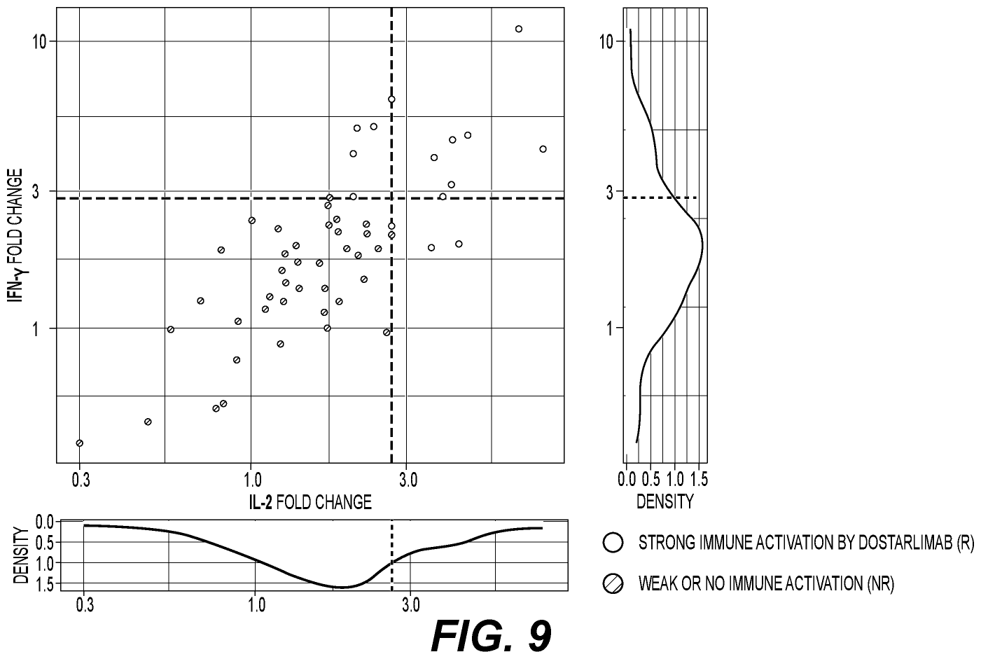

NSCLC tumors were grouped according to their level of immune activation after ex vivo treatment with dostarlimab. Fold changes in secreted IFN-γ and IL-2 were used as a measure of intratumoral immune activation. The top 28% of tumors with the strongest FC in IL-2 or IFN-γ were grouped as “strong immune activated by dostarlimab” (N=15), while the remaining 72% (N=39) were grouped as “weak or no immune activation” (FIG. 9).

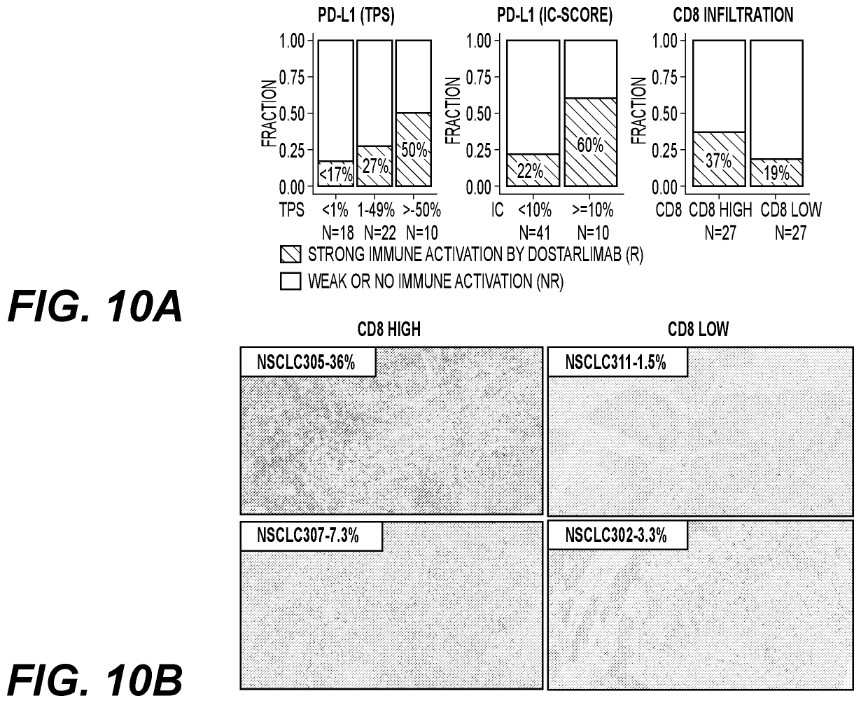

An increase in immune activation was observed in tumors with higher PD-L1 expression and CD8 infiltration following treatment with dostarlimab (FIG. 10). Specifically, immune activation in the PDE model was proportional to the percentage of CD8+ T-cells present in the tumor tissue (Table 6). Significant correlations were observed between IFN-γ median fold change (FC) and PD-L1 TPS and IC scores as well as CD8+ T-cell infiltration. Significant correlation was further observed between IL-2 median FC and PD-L1 IC score and CD8+ T-cell infiltration.

TABLE 6
Correlation analysis (Pearson or Student's T-test)
between tumor or donor features and the median fold
change of secreted IFN-γ and IL-2 in the PDE model.

| | | p-value | p-value |
|---|---|---|---|
| | | for IFN-γ | for IL-2 |
| Feature | test | median FC | median FC |
| TPS score PD-L1 | Pearson | 5.45 e−04* | 3.88 e−02 |
| IC score PD-L1 | Pearson | 2.49 e−04* | 5.06 e−03* |
| CD8+ infiltration | Pearson | 2.54 e−07* | 1.30 e−03* |
| CD3+ infiltration | Pearson | 0.200 | 0.387 |
| TMB | Pearson | 0.498 | 0.321 |
| Diagnosis (LUAD/LUSC) | Student's T-test | 0.235 | 0.525 |
| Gender | Student's T-test | 0.750 | 0.761 |
| Grading | Student's T-test | 0.175 | 0.411 |

*p < 0.01 correlation between feature and cytokine release

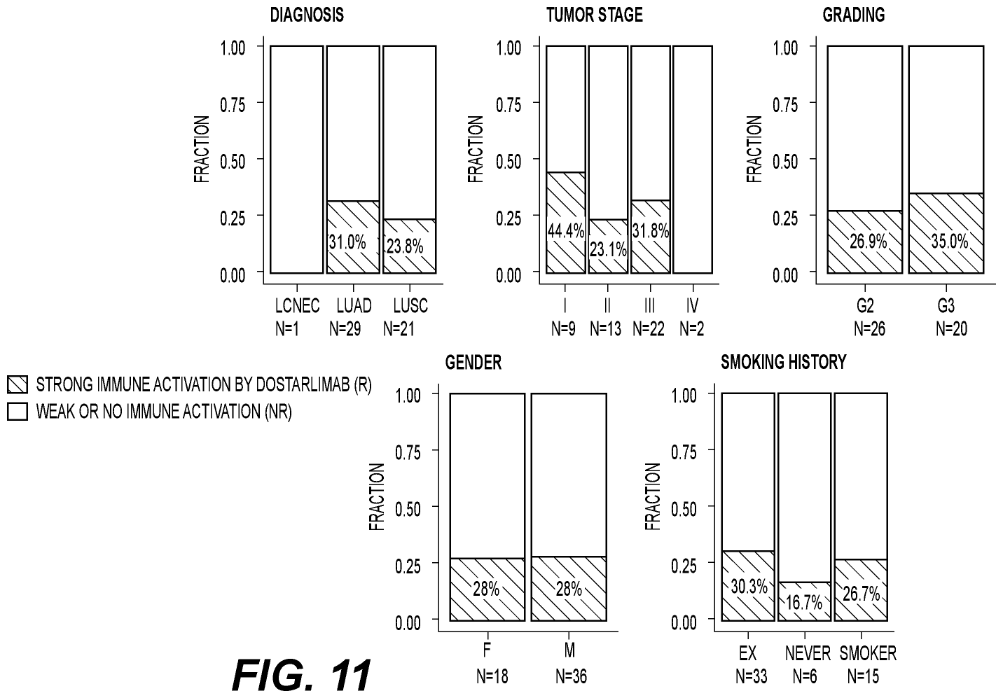

The donor and tumor characteristics of responders (R) and non-responders (NR) to dostarlimab in the NSCLC PDE model are shown in FIG. 11. The group of tumors with strong activation after treatment with dostarlimab distributed similarly in the two main diagnosis groups. LUAD and LUSC. No correlation was observed between gender or smoking status and immune activation in the PDE NSCLC model. The level of immune activation in response to dostarlimab ex vivo treatment was balanced between male and female, as well as between the different smoking status of the NSCLC tumors that were tested in the PDE model. In tumor stage and grading, no major differences in the proportion of the two immune activation groups, i.e., strong vs. weak/no immune activation by dostarlimab) were observed.

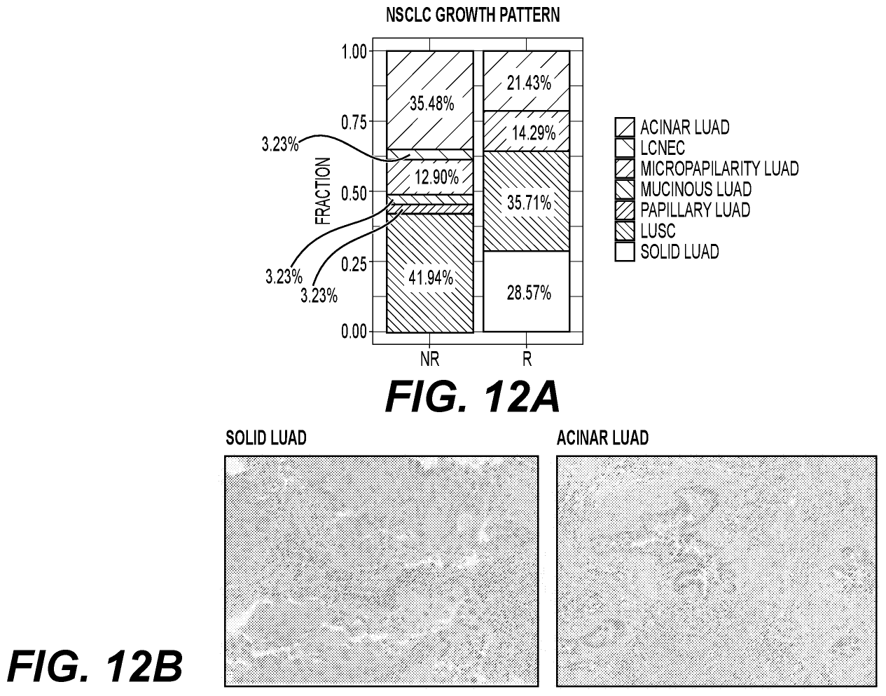

The fraction of responders (R) and non-responders (NR) to dostarlimab in the PDE model in different types of NSCLC growth patterns is shown in FIG. 12. The results show that immune activation is heterogenous across different NSCLC subtypes. In the cohort of N=45 NSCLC donors for which NSCLC subtype information was available, all solid LUAD were grouped into the tumors showing a strong immune activation by dostarlimab. This is in line with studies showing that solid LUAD have higher PD-L1 expression (Dong et al., J Thorac Oncol. 2018, 13(1):85-96; Driver et al., Arch Pathol Lab Med. 2017, 141(11):1529-1532; Beshai et al., Int J Cancer Clin Res. 2021, 8:154) and thus have higher chances to benefit from immune checkpoint inhibition. Surprisingly, acinar LUAD was more represented in the group of weak or no responders.

### Identification of Predictive Biomarkers for Response to Dostarlimab Treatment:

The baseline tumor immune microenvironment was phenotyped using a 39-color spectral flow cytometry panel. The linear association between cytokine production in PDE and immune activation of over 30 immune cells subtypes was modelled in N=16 NSCLC tumors. A linear mixed model with cytokine production as a continuous explanatory variable was fitted, also accounting for a random batch effect due to the data being acquired in batches.

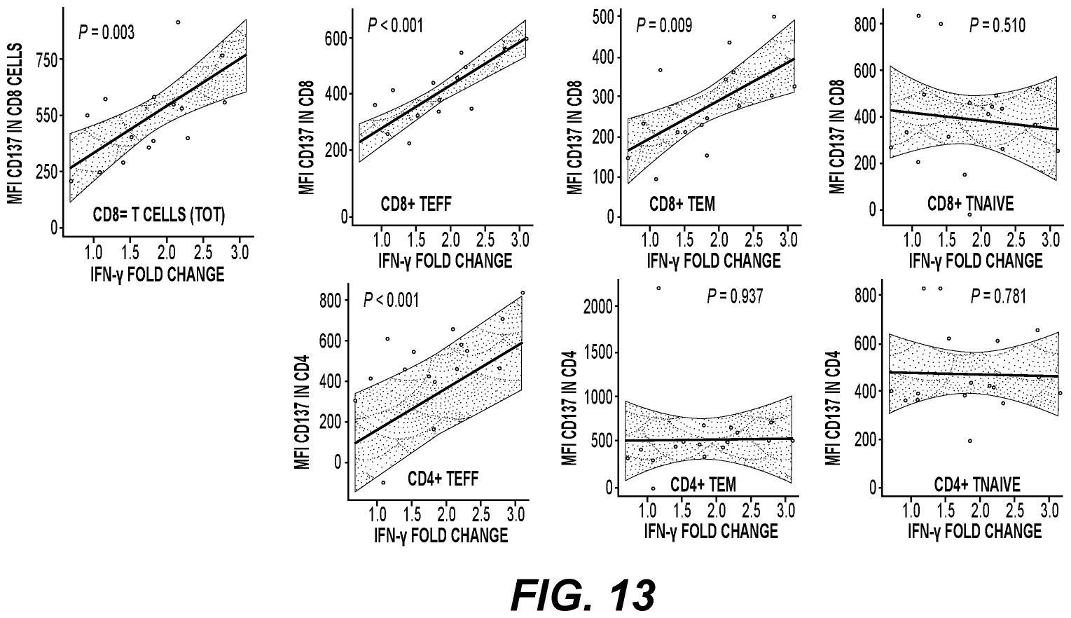

Baseline expression of CD137 in specific CD8+ and CD4+ T cells subtypes correlated with immune activation in response to dostarlimab treatment in the NSCLC PDE model (FIG. 13). Median fluorescence intensity (MFI) of CD137 in CD8+ T-cells was positively associated (p<0.01) with the median fold change of secreted IFN-γ. Positive association between the CD137 MFI and IFN-γ was also significant for the effector (Teff) and effector memory (Tem) subtypes of CD8+ T cells, but not for the naïve CD8+ T cells. Similarly, CD137 expression correlated with immune activation in CD4+ Teff, but not in naïve CD4 cells. These results are in line with the observation that high levels of circulating CD137+ T-cells are associated with a prolonged PFS and OS to nivolumab or pembrolizumab (Zizzari et al., Clin Cancer Res. 2022, 28(5):1027-1037; Ugolini et al., EBioMedicine, 2020, 62:103098).

### Example 3: Treatment of Non-Small Cell Lung Cancer (NSCLC) with Dostarlimab in a Patient Previously Treated with Pembrolizumab Plus Chemotherapy

An NSCLC patient previously treated with pembrolizumab plus chemotherapy is switched to receiving treatment with dostarlimab or a biosimilar thereof, optionally in combination with chemotherapy. In some cases, the patient's treatment is switched because the patient demonstrates progressive disease after undergoing treatment with pembrolizumab or a biosimilar thereof plus chemotherapy. In some cases, the patient's treatment is switched because the patient demonstrates only stable disease after undergoing treatment with pembrolizumab or a biosimilar thereof plus chemotherapy. In some cases, the patient's treatment is switched because the patient demonstrates anti-pembrolizumab antibodies. After switching to treatment with dostarlimab or a biosimilar thereof, the patient demonstrates an improved response.

### Example 4: Randomized Phase 3, Open-Label Study of Dostarlimab as Sequential Therapy in Patients with Locally Advanced, Unresectable Non-Small Cell Lung Cancer (Stage III) Who have not Progressed Following Definitive, Platinum-Based, Concurrent Chemoradiation Therapy Versus Durvalumab

A randomized, open-label Phase 3 trial to evaluate the effect of dostarlimab compared to durvalumab as consolidation therapy in participants with locally advanced unresectable NSCLC (LA NSCLC) who have received at least 2 cycles of definitive, platinum based, concurrent chemoradiotherapy and have not progressed. The primary objective is to evaluate PFS of dostarlimab compared to durvalumab, where PFS is defined as the proportion of patients free from radiographic progression according to BICR based on RECIST 1.1, or death due to any cause after treatment randomization, whichever occurs first. A key secondary objective is to evaluate OS of dostarlimab compared to durvalumab, where OS is defined as the time from treatment randomization until death due to any cause. Eligible participants will have pathologically confirmed, locally advanced unresectable Stage III NSCLC, and have received at least 2 cycles of definitive, platinum based, concurrent chemoradiotherapy and have not progressed will be randomized 1:1 via IVRS to receive either dostarlimab 1000 mg Q6W, within a treatment period of up to 12 months (Arm A), or durvalumab 1500 mg Q4W up to 12 months (Arm B). Study intervention must be discontinued early in the event of disease progression or intolerable toxicity. Randomization is ideally performed on C1D1 prior to first study intervention but can be within −3 days of C1D1 for logistic reasons. All timepoints for assessments during the treatment and follow-up periods will be anchored to the day of randomization, if different from the day of first study drug dose (C1D1).

Following completion of the 12-month treatment period, or treatment discontinuation for any reason, participants will enter the Follow-Up phase which includes an EoT Visit within 30 days after last dose of study treatment and a safety follow-up visit at 90 days after last dose of study treatment. Participants will be followed for adverse events AEs/serious adverse events (AEs/SAEs) throughout the treatment period through 90 days after last dose of study treatment or until the subject starts alternate anticancer therapy, whichever occurs first. SAEs assessed as related to study participation or to study drugs will be recorded from the time a participant consents to participate in the study. SAEs assessed as related to pre-study participation will be recorded from the time a participant consents for the optional pre-screening blood sample through 24 hours after the procedure.

During the Follow-Up period, patients will continue to be followed for the occurrence of a primary endpoint defining event including death (if not already occurred), routine safety laboratory assessments, and subsequent anti-cancer therapy. Participants considering study withdrawal will be offered a modified follow-up option to stay in the study for collection of survival data only. Participants permanently discontinuing study intervention prior to documented disease progression verified by BICR as per RECIST v1.1 will be followed by imaging as per schedule of activities on their original calendar schedule. Participants discontinuing study intervention due to disease progression will be followed according to Follow-up schedule.

A total of approximately 862 participants (approximately 431 per treatment arm) will be enrolled. The study has four planned analyses. The primary analysis of PFS will occur once 479 PFS events are accumulated, approximately four years from study start. The final OS analysis is planned at 379 OS events.

TABLE 7
Randomization Schema

| | Number of | | Study |
|---|---|---|---|
| Study Arms | Participants | Age | Interventions |
| Arm A: Dostarlimab | 431 | 18+ | IV infusion |
| Arm B: Durvalumab | 431 | | IV infusion |

TABLE 8
Study Interventions to be Administered

| | Dostarlimab | Durvalumab |
|---|---|---|
| Study intervention name: | Dostarlimab | Durvalumab |
| Unit dose strength | 1000 mg Q6W | 1500 mg Q4W |
| Route of administration | IV infusion | IV infusion |
| No of doses | Up to 9 | Up to 13 |
| Type | Intervention | Control |

Dostarlimab will be administered through a 30 minute infusion at a dose of 1000 milligrams (mg) intravenously (IV) every 6 weeks (Q6W) up to a maximum of 9 cycles (each cycle of 42 days) for study subjects randomized to Arm A.

Durvalumab will be administered through a 60 minute infusion at a dose of 1500 milligrams (mg) intravenously (IV) every 4 weeks (Q4W) up to a maximum of 13 cycles (each cycle of 28 days) for study subjects randomized to Arm B.

The final statistical analysis will demonstrate that dostarlimab performs at least as well as durvalumab in the ITT patient population.

### Example 5: PD-1 Jurkat and PD-L1 CHO Single Receptor Cellular Assay

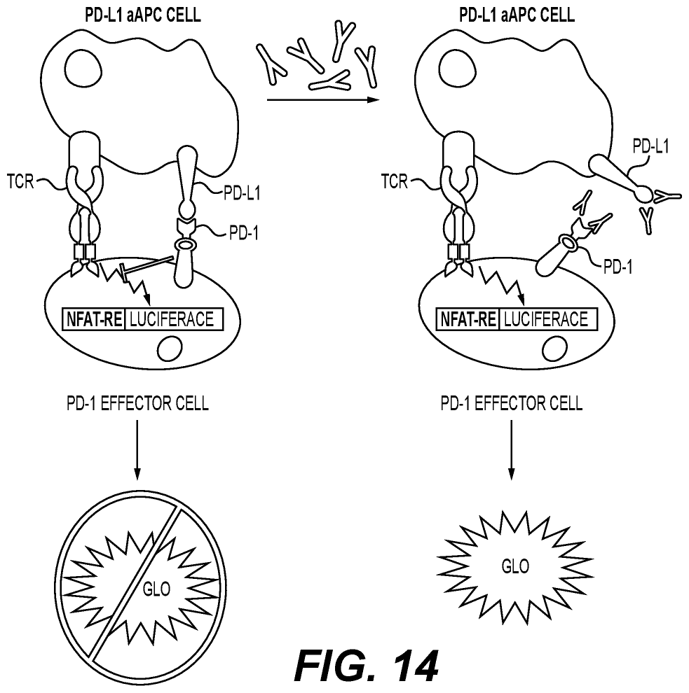

A cell-based, in vitro, assay was conducted to monitor the effect of interfering with with PD-1::PD-L1 cellular signaling. The overall assay schema is shown in FIG. 14. Jurkat cells were used for the PD-1 Effector cells and Chinese Hamster Ovary (CHO) cells were used for the PD-L1 antigen presenting cells (APC).

Jurkat PD-1 Effector cells and CHO-PDL1 (PD L1 APC/CHO-K1) cells were obtained from Promega (Madison, WI). Cell culturing reagents were obtained from Life Technologies. Jurkat PD-1 Effector cells were routinely cultured in T150 flasks at 37° C. and 5% CO₂ in RPMI-1640 supplemented with 10% fetal bovine serum (FBS), 500 μg/mL Hygromycin B and 100 μg/mL Geneticin (G418). Jurkat PD-1 cells were grown between 0.3 and 1.9×10⁶ cells/ml and passaged by dilution in supplemented RPMI-1640. CHO-PDL1 cells were routinely cultured in T150 flasks at 37° C. and 5% CO₂ in F12 supplemented with 10% FBS, 250 μg/mL Hygromycin B and 200 μg/mL Geneticin.

The CHO-PDL1 cells were harvested at approximately 80% confluency using Versene. The cells were resuspended in RPMI-1640+2% FBS (R2). Cells were counted (Vi-Cell XR Cell Viability Analyzer) and a 1× cell stock was prepared at 0.5×10⁶ cells/mL in R2 media. Cells were added to 1536-well white tissue-culture treated plates (Greiner) at a density of 1000 cells/well (2 μL) using the BioTek EL406 liquid dispenser.

The PD-1 Jurkat Effector cells were resuspended in 10 mL R2 media. The cells were counted, and a 0.5×10⁶ cells/mL cell solution (IX) was prepared in R2 media. Effector cells were added to the CHO-PDL1 cell plates at a density of 1000 cells/well (2 μL) using the BioTek liquid dispenser for a total volume of 4 μL per well. The final Jurkat to CHO cell ratio was 1:1. Antibodies were dosed using the ECHO 555 Acoustic Liquid Handler (Labcyte). The ECHO 555 was used to add antibody from the source plate in a 10 point dose titration from 14.56 μg/mL to 0.00036 μg/mL final concentration. PBS was added to reach a total volume of 60 nL. Ten replicates/concentration were evaluated for each antibody.

To form working solutions. Dostarlimab (TSR-042, Lot 285SI50327Y14), Keytruda (Merck, NDC 0006-3026-02), Opdivo (BMS, NDC 0003-3734-13) and IGg4 (Eureka, ET904) antibodies were diluted to 1000, 100, 10, 1, and 0.1 μg/mL in Dulbecco's phosphate buffered saline (PBS) in a 96-well plate (Falcon, Cat #351177). Fifty μL of each dilution was then added to an ECHO Qualified 384-well polypropylene source plate (Labcyte, Cat #PPT-0200). PBS alone was used for backfilling. Unique antibody plates were prepared for the N=1 and N=2 studies and stored at 4° C. until needed.

Assay plates were incubated for 24 hours at 37° C. and 5% CO₂. Plates were brought to room temperature and Bio-Glo Luciferase Assay system reagent (Promega, cat #G7940) was prepared according to manufacturer's instructions. Three μL of Bio-Glo luciferase reagent was added to each well using the Bio-Tek dispenser. Plates were incubated at room temperature for 5 minutes and luminescence was read on an EnVision (Perkin Elmer) plate reader. Data was graphed as RLU versus Antibody concentration (μg/ml) in GraphPad Prism (version 7.02) and curves were fit with the 3-parameter fit program.

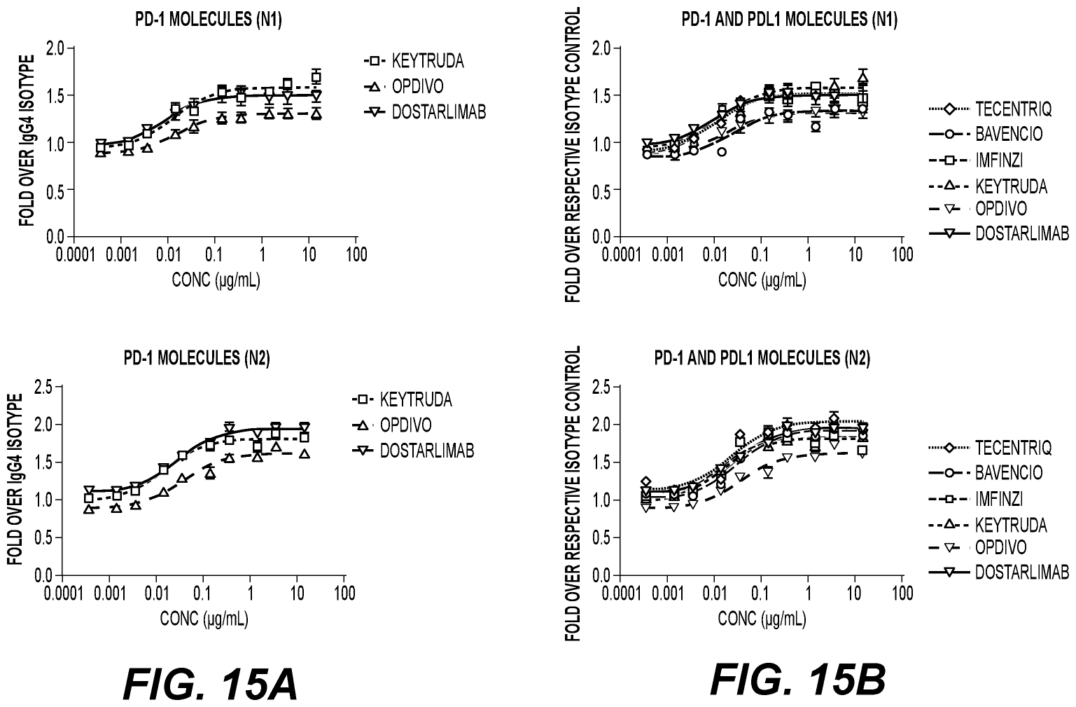

FIG. 15 shows the results. The fold response above the IgG4 isotype control (negative control) is plotted in each panel (A and B). FIG. 15A shows the titration curve for the anti-PD-1 molecules: Keytruda (pembrolizumab), Opdivo (nivolumab), and dostarlimab (Jemperli). Each anti-PD-1 mAb shows a dose-response curve in this PD-1/PD-L1 cell-based functional assay. FIG. 15B shows the data for both the anti-PD-1 and the anti-PD-L1 mAbs. The anti-PD-1 mAbs are Keytruda (pembrolizumab), Opdivo (nivolumab), and dostarlimab (Jemperli) and the anti-PD-L1 mAbs are Tencentriq (atezolizumab), Bavencio (avelumab), and Imfinzi (durvalumab). Each anti-PD-1 mAb and each anti-PD-L1 mAb shows a dose-response curve in this PD-1/PD-L1 cell-based functional assay. Each show a dose positive dose-response compared to the isotype control.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, the group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Certain embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of treating non-small cell lung cancer (NSCLC) in a patient previously treated with a combination of (i) an anti-PD-1 antibody or an anti-PDL1 antibody that is not dostarlimab and (ii) one or more chemotherapeutic agents, the method comprising administering a therapeutically effective amount of dostarlimab to the patient, wherein the patient discontinued administration of the immune checkpoint inhibitor prior to beginning administration of dostarlimab.

2. (canceled)

3. (canceled)

4. The method of claim 1, wherein the anti-PD-1 antibody is selected from the group consisting of pembrolizumab, nivolumab, and cemiplimab.

5. The method of claim 4, wherein the anti-PD-1 antibody is pembrolizumab.

6. (canceled)

7. The method of claim 1, wherein the anti-PD-L1 antibody is selected from the group consisting of atezolizumab, avelumab, and durvalumab.

8.-27. (canceled)

28. The method of claim 1, wherein the dostarlimab is administered at a dose of 500 mg.

29. The method of claim 28, wherein the dostarlimab is administered through a 30 minute infusion intravenously Q3W.

30. The method of claim 29, wherein the dostarlimab is administered at a dose of 1,000 mg beginning 3 weeks after dose 4.

31. The method of claim 30, wherein the dostarlimab is administered every 6 weeks.

32.-37. (canceled)

38. The method of claim 1, wherein the NSCLC is metastatic NSCLC.

39.-40. (canceled)

41. The method of claim 1, wherein the patient has a PD-L1 tumor proportion score (TPS) of 50% or greater.

42. A method of treating non-small cell lung cancer (NSCLC) in a patient previously treated with a combination of pembrolizumab, and one or more chemotherapeutic agents, the method comprising administering a therapeutically effective amount of dostarlimab, to the patient.

43. (canceled)

44. The method of claim 42, wherein the NSCLC is metastatic NSCLC.

45.-75. (canceled)

76. The method of claim 1, wherein said administration results in improved objective response rate (ORR) relative to administration of the immune checkpoint inhibitor that is not dostarlimab, and the one or more chemotherapeutic agents.

77. The method of claim 1, wherein the method demonstrates non-inferiority to administration of the immune checkpoint inhibitor that is not dostarlimab, and the one or more chemotherapeutic agents.

78. The method of claim 77, wherein non-inferiority is demonstrated using objective response rate (ORR).

79.-90. (canceled)

91. A method of treating non-small cell lung cancer (NSCLC) in a patient previously treated with chemotherapy and radiotherapy comprising administering dostarlimab or a biosimilar thereof 500 mg Q3W.

92. The method of claim 91 wherein the dostarlimab or a biosimilar thereof is administered 1000 mg Q6W.

93. The method of claim 91 wherein the patient has been previously treated with an anti-PD-L1 antagonist.

94. (canceled)

95. (canceled)
